(19) United States
(12) Patent Application Publication
(10) Pub. No.: US 2025/0057752 A1
(43) Pub. Date: Feb. 20, 2025

(54) METHODS FOR EXTRACTING ALGINATE BIOMASS AND RELATED COMPOSITIONS

(71) Applicant: C-Combinator, a Public Benefit Corporation, Boston, MA (US)

(72) Inventors: Taili Thula, Guaynabo, PR (US); Lisbeth E. Echevarria Núñez, San Juan, PR (US); Emanuel Fonseca Dinis Costa, Guarda (PT); Benjamin Ivan Jelen, Austin, TX (US); Wilfredo Carlos Boyrié Delgado, Ponce, PR (US); Hector Luis Paris Oliveras, Canovanas, PR (US)

(21) Appl. No.: 18/721,930

(22) PCT Filed: Dec. 22, 2022
(86) PCT No.: PCT/US22/53840

§ 371 Date: Jun. 20, 2024

Related U.S. Application Data
(60) Provisional application No. 63/293,269, filed on Dec. 23, 2021.

(51) Int. Cl. A61K 8/73; A61K 8/06; A61Q 19/00; C08B 37/00

(52) U.S. Cl. CPC A61K 8/733; A61K 8/062; A61Q 19/00; C08B 37/0084

(57) ABSTRACT

The disclosure relates to methods for extracting an alginate biomass as well as related extract compositions. A biomass pulp is subjected to a multi-step or multi-stage extraction process including a first alkaline extraction followed by a second water or dilution extraction. The first extraction involves contacting the biomass pulp with a base such as a carbonic acid salt to form a biomass dough and convert alginate present in the biomass to a water-soluble alginate salt. The second extraction involves adding water to the biomass dough to form a biomass suspension, followed by partitioning and separation to form separate biosolids and bioliquids phases. Both extraction steps are performed at mild temperatures to preserve high-molecular weight polymer complexes including alginate and protein materials extracted from the biomass. The biosolids, bioliquids, and corresponding polymer complexes have favorable emulsifier properties, for example for use in cosmetic and other formulations.

### CROSS REFERENCE TO RELATED APPLICATION

Priority is claimed to U.S. Provisional Application No. 63/293,269 (filed Dec. 23, 2021), which is incorporated herein by reference in its entirety.

### STATEMENT OF GOVERNMENT INTEREST

None.

### BACKGROUND OF THE DISCLOSURE

### Field of the Disclosure

The disclosure relates to methods for extracting an alginate biomass as well as related extract compositions. A biomass pulp is extracted in sequential processes using a base such as a carbonic acid salt and water at mild temperatures to partition, separate, and recover biosolids and bioliquids phases having high-molecular weight polymer complexes including alginates and proteins, which have useful emulsifier properties.

### SUMMARY

In one aspect, the disclosure relates to a method for extracting an alginate biomass, the method comprising: providing an alginate-containing biomass pulp; performing a first (alkaline) extraction process comprising: adding a base such as a carbonic acid salt (e.g., with a monovalent or divalent cation for solubilizing alginate and optionally other polysaccharides) and optionally water (e.g., depending on water content of biomass pulp) in amounts sufficient to the biomass pulp to provide a biomass dough having (i) a water content in a range of 40-80 wt. %, (ii) a base content in a range of 5-20 wt. %, and (iii) a solids (e.g., biomass solids) content in a range of 15-55 wt. % based on the biomass dough; and partitioning or maintaining the biomass dough at a temperature up to 35° C. (e.g., 15-30° C. or about room/ambient temperature) for a time sufficient to convert alginate originally present in the biomass pulp (e.g., in alginic acid form, insoluble alginate salt form, and/or soluble alginate salt form) into a water-soluble alginate salt (e.g., sodium alginate dissolved in the water portion of the biomass dough); and performing a second (water or dilution) extraction process comprising: adding water in an amount sufficient to the biomass dough containing the water-soluble alginate salt to provide a biomass suspension having (i) a water content in a range of 80-98 wt. % (e.g., and higher than that of the biomass dough), and (ii) a solids content in a range of 2-20 wt. % based on the biomass suspension; and maintaining the biomass suspension at a temperature up to 35° C. (e.g., 15-30° C. or about room/ambient temperature) for a time sufficient to form a bioliquids phase and a separate biosolids phase therein, wherein (i) the bioliquids phase comprises water and at least a portion of the water-soluble alginate salt dissolved therein, and (ii) the biosolids phase comprises proteins, optionally amino acids, cellulose, and at least a portion of the water-soluble alginate salt extracted from the biomass pulp. Suitably, the extraction method further comprises separating the bioliquids phase from the biosolids phase (e.g., via centrifugation, settling, or other gravimetric, mechanical, or physical separation process).

In another aspect, the disclosure relates to a method for extracting an alginate biomass, the method comprising: providing an alginate-containing biomass pulp; performing a first (alkaline) extraction process comprising: adding a base selected from the group consisting of inorganic acid salts and hydroxides, and optionally water in amounts sufficient to the biomass pulp to provide a biomass dough; and maintaining the biomass dough at a first temperature for a time sufficient to convert alginate originally present in the biomass pulp into a water-soluble alginate salt; and performing a second (water or dilution) extraction process comprising: adding water in an amount sufficient to the biomass dough containing the water-soluble alginate salt to provide a biomass suspension; and partitioning the biomass suspension at a second temperature for a time sufficient to form a bioliquids phase and a separate biosolids phase therein, wherein (i) the bioliquids phase comprises water and at least a portion of the water-soluble alginate salt dissolved therein, and (ii) the biosolids phase comprises proteins, optionally amino acids, cellulose, and at least a portion of the water-soluble alginate salt extracted from the biomass pulp. Suitably, one or both of the first temperature of the first extraction and the second temperature of the second extraction is 35° C. or less. Suitably, the biomass dough has a water content of at least 40 wt. % (e.g., 40-90 wt. % or 40-80 wt. %). Suitably, the biomass suspension has a water content of at least 70 wt. % (e.g., 70-95 wt. % or 80-98 wt. %).

Various refinements of the disclosed methods and related compositions are possible.

In refinements, the base is selected from the group consisting of inorganic acid salts (e.g., carbonic acid salt) and hydroxides, for example metal or ammonium salts or hydroxides. The inorganic acid salt can be sodium carbonate. More generally, the inorganic acid salt can comprise an anion selected from the group consisting of carbonate, bicarbonate, sulfate, bisulfate, nitrate, phosphate, hydrogen phosphate, and dihydrogen phosphate; and a cation selected from the group consisting of alkali metal cations, alkaline earth metal cations, and ammonium. Alternatively, the base can be selected from the group consisting of alkali metal hydroxides, alkaline earth metal hydroxides, and ammonium hydroxide.

In refinements, the bioliquids phase is 50 to 90 wt. % of the biomass suspension; and/or the biosolids phase is 10 to 50 wt. % of the biomass suspension (e.g., where bioliquids and biosolids sum to at least 95, 98, 99, or 100 wt. %).

In refinements, the biosolids phase has a water content in a range of 84 to 96 wt. % relative to the biosolids phase (i.e., wet-weight or total basis); the biosolids phase has an inorganic content in a range of 2 to 8 wt. % relative to the biosolids phase; and/or the biosolids phase has an organic content in a range of 2 to 8 wt. % relative to the biosolids phase, for example where water, inorganics, and organics sum to at least 95, 98, 99, or 100 wt. %. The total solids as a sum of inorganics and organics (e.g., dissolved, suspended, complexed, etc.) can be in a range of 4 to 16 wt. %.

In further refinements, the biosolids phase has an inorganic content in a range of 20 to 80 wt. % on a dry weight basis (i.e., excluding water content, but including all solids whether in dissolved, suspended, or complexed form); and the biosolids phase has an organic content in a range of 20 to 80 wt. % on a dry weight basis (e.g., inorganics and organics sum to 100 wt. % on dry weight basis).

In further refinements, the biosolids phase comprises: proteins in an amount in a range of 5 to 30 wt. % relative to total organic content; and/or free amino acids. Total amino acids between the proteins and the free amino acids (when present) can comprise at least one of glutamic acid, aspartic acid, glycine, leucine, alanine, and arginine (e.g., multiple acids or all in combination). For example, the total amino acids can comprise aspartic acid and alanine; and the aspartic acid and the alanine can be present in a combined amount in a range of 15 to 25 wt. % relative to the total amino acids (e.g., in the biosolids phase). Similarly, the total amino acids can comprise glutamic acid, aspartic acid, glycine, leucine, alanine, and arginine; and the glutamic acid, the aspartic acid, the glycine, the leucine, the alanine, and the arginine can be present in a combined amount in a range of 40 to 60 wt. % relative to the total amino acids (e.g., in the biosolids phase).

In refinements, the extraction method further comprises performing at least one of drying and crosslinking of the separated biosolids phase.

In refinements, the bioliquids phase has a water content in a range of 90 to 99 wt. % relative to the bioliquids phase (i.e., wet-weight or total basis); the bioliquids phase has an inorganic content in a range of 0.5 to 9 wt. % relative to the bioliquids phase; and the bioliquids phase has an organic content in a range of 0.5 to 9 wt. % relative to the biosolids phase, for example where water, inorganics, and organics sum to at least 95, 98, 99, or 100 wt. %. The total solids as a sum of inorganics and organics (dissolved, suspended, complexed, etc.) can be 1 to 10 wt. %.

In further refinements, the bioliquids phase has an inorganic content in a range of 20 to 80 wt. % on a dry weight basis (i.e., excluding water content, but including all solids whether in dissolved, suspended, or complexed form); and the bioliquids phase has an organic content in a range of 20 to 80 wt. % on a dry weight basis (e.g., inorganics and organics sum to 100 wt. % on dry weight basis).

In refinements, the extraction method further comprises removing at least a portion of the water from the bioliquids phase (e.g., via drying or otherwise) to form a dried bioliquids.

In refinements, at least one of the biosolids phase and the bioliquids phase comprises a polymer or biopolymer complex comprising alginate, protein, and optionally cellulose bound together. In further refinements, the polymer complex can have a peak molecular weight or an average molecular weight (e.g., number-average or weight-average) in a range of about 400 kDa to 3000 kDa or 30 kDa to 3000 kDa.

In refinements, the extraction method further comprises precipitating the water-soluble alginate salt.

In refinements, the alginate-containing biomass pulp comprises sargassum pulp.

In refinements, providing the alginate-containing biomass pulp comprises one or more of: drying and size-reducing an alginate-containing biomass; performing a peroxide treatment or other decoloring treatment (e.g., UV, bleach, or chloroform treatment) on the alginate-containing biomass; and/or performing an acidification treatment on the alginate-containing biomass.

In refinements, providing the alginate-containing biomass comprises performing a treatment on the alginate-containing biomass selected from the group consisting of a peroxide treatment, a UV treatment, a bleach treatment, and a chloroform treatment. In a further refinement, the peroxide treatment is performed, and the peroxide treatment can comprise contacting the alginate-containing biomass with hydrogen peroxide in an amount from 0.5 wt. % to 50 wt. % relative to the alginate-containing biomass. Alternatively, the peroxide treatment is performed, and the peroxide treatment can comprise contacting the alginate-containing biomass with hydrogen peroxide in an amount from 0.5 wt. % to 250 wt. % relative to the alginate-containing biomass.

In refinements, the extraction method comprises adding the base (e.g., carbonic acid salt) in solid form to the biomass pulp.

In refinements, the extraction method comprises performing the first extraction process at a pH in a range of 1.5 to 11.

In refinements, the method further comprises combining (e.g., via high shear mixing) at least one of the biosolids phase (or a portion thereof) and the bioliquids phase (or a portion thereof) with a thickening agent and optionally a preservative, thereby forming an emulsifier composition. In a further refinement, the emulsifier composition comprises the biosolids phase, which is a sargassum extract and is present in an amount in a range of 80 to 95 wt. % (or 80 to 90 wt. %) relative to emulsifier composition; the thickening agent comprises xanthan gum, which is present in an amount in a range of 3 to 15 wt. % (or 7 to 15 wt. %) relative to emulsifier composition; and the preservative is present and comprises pentylene glycol, which is present in an amount in a range of 1 to 8 wt. % (or 3 to 8 wt. %) relative to emulsifier composition.

In another aspect, the disclosure relates to a biosolids material according to the any of the variously disclosed embodiments herein, for example a biosolids material formed by any of the disclosed extraction and subsequent processing methods. For example, the biosolids material can include the biosolids phase after separation from the bioliquids phase in the biomass suspension (e.g., but prior to any further processing). Alternatively, the biosolids material can include the biosolids obtained after subsequent processing of the biosolids phase, such as by one or more of drying, crosslinking, etc.

In another aspect, the disclosure relates to a bioliquids material according to the any of the variously disclosed embodiments herein, for example a bioliquids material formed by any of the disclosed extraction and subsequent processing methods. For example, the bioliquids material can include the bioliquids phase after separation from the biosolids phase in the biomass suspension (e.g., but prior to any further processing). Alternatively, the bioliquids material can include the bioliquids obtained after subsequent processing of the bioliquids phase, such as by one or more of drying, crosslinking, precipitation, etc.

In another aspect, the disclosure relates to an emulsifier composition comprising: at least one of a biosolids alginate biomass extract and a bioliquids alginate biomass extract; a thickening agent; and optionally a preservative.

Various refinements of the disclosed emulsifier compositions are possible.

In refinements, the emulsifier composition comprises the biosolids alginate biomass extract; and the biosolids alginate biomass extract is present in an amount in a range of 50 to 98 wt. % (or 80 to 90 wt. %) relative to the emulsifier composition.

In refinements, the thickening agent is present in an amount in a range of 2 to 40 wt. % (or 5 to 20 wt. %) relative to the emulsifier composition; and the thickening agent is selected from the group consisting of xanthan gum, guar gum, konjac gum, sclerotium gum, cellulose and cellulose gum, carrageenan, alginates plus crosslinker, crosslinked sargassum bioliquid extracts, carbomers, cetearyl alcohol, glycerol stearate, glycerol stereate citrate, polyacrylates, hydroxyethylcellulose, and combinations thereof.

In refinements, the preservative is present in an amount in a range of 1 to 20 wt. % (or 2 to 10 wt. %) relative to the emulsifier composition; and the preservative is selected from the group consisting of pentylene glycol, phenoxyethanol, sodium levulinate, potassium sorbate, honeysuckle extract, caprylhydroxamic acid, sodium anisate, propylene glycol (or propanediol), diazolidinyl urea, iodopropynyl butylcarbamate, ethanol, and combinations thereof.

In refinements, the emulsifier composition comprises the biosolids alginate biomass extract, which is a sargassum extract and is present in an amount in a range of 80 to 95 wt. % (or 80 to 90 wt. %) relative to the emulsifier composition; the thickening agent comprises xanthan gum, which is present in an amount in a range of 3 to 15 wt. % (or 7 to 15 wt. %) relative to the emulsifier composition; and the preservative is present and comprises pentylene glycol, which is present in an amount in a range of 1 to 8 wt. % (or 3 to 8 wt. %) relative to the emulsifier composition.

In refinements, the alginate biomass extract comprises a sargassum biomass extract; and the alginate biomass extract comprises a polymer complex comprising alginate, protein, and optionally cellulose bound together (e.g., having a peak, number-average, or weight-average molecular weight in a range of 400 kDa to 3000 kDa or 30 kDa to 3000 kDa).

In refinements, the alginate biomass extract is a biosolids alginate biomass extract or portion/fraction thereof comprising a biosolids material formed according to the disclosed extraction process in any of its various embodiments, refinements, etc.

In refinements, the alginate biomass extract is a bioliquids alginate biomass extract or portion/fraction thereof comprising a bioliquids material formed according to the disclosed extraction process in any of its various embodiments, refinements, etc.

In refinements, the emulsifier composition has a pH value in a range of 7 to 11 (or 8 to 9.5).

In refinements, the emulsifier composition has a viscosity value in a range of 200 to 2000 cP (or 400 to 1000 cP), as measured in a 10 wt. % aqueous solution of the emulsifier composition at 25° C.

In another aspect, the disclosure relates to an emulsion (e.g., an oil-in-water emulsion) comprising: water (e.g., as a primary component of an aqueous phase, such as a continuous aqueous phase); oil (e.g., as a dispersed phase in the continuous aqueous phase); and the emulsifier composition according to any of the variously disclosed embodiments (e.g., at an interface between the water/aqueous phase and the oil phase to stabilize the emulsion).

Various refinements of the disclosed emulsion compositions are possible.

In refinements, the emulsion is in the form of an oil-in-water emulsion; the water is present in an amount in a range of 50 to 98 wt. % relative to the emulsion; the oil is present in an amount in a range of 2 to 50 wt. % relative to the emulsion; and/or the emulsifier composition is present in an amount of 0.2 to 20 wt. % relative to the emulsion.

In refinements, a ratio of emulsifier composition to oil (or total oil) present in the emulsion is in a range of 1:1.5 to 1:10 (w/w) (or 1:2 to 1:8, 1:3 to 1:7, or 1:4 to 1:6, reflecting an excess of oil relative to emulsifier composition).

In refinements, the oil comprises an emollient selected from the group consisting of caprylic/capric triglyceride, coco caprylate caprate, mineral oil, octyldodecanol, coconut oil, grapeseed oil, sunflower oil, squalane, silicones (e.g., dimethicone, silicone quarternium-3, trideceth-12, amodimethicone, c11-15 alketh-12, c11-15 alketh-7, cyclopentasiloxane), avocado oil, olive oil, jojoba oil, almond oil, sunflower wax, beeswax, shea butter, ethers (e.g., dicaprylyl ether), esters (e.g., cetyl esters, ethyl palmate, isopropyl palmitate, octyl pamitate, jojoba esters, octyldodecyl oleate), limnanthes alba (meadowfoam) seed oil, and combinations thereof.

In refinements, the oil has a polarity index in a range of 15 mN/m to 50 mN/m or 15 mN/m to 35 mN/m.

In refinements, the emulsion comprises one or more further components (or additives) selected from the group consisting of preservatives, thickening agents, chelating agents, anti-oxidants, active agents, surfactants, pigments, colorants, UV filters, pH-adjusting agents (e.g., acids, bases, buffers), and combinations thereof.

In refinements, the emulsion has a pH value in a range of 3 to 11 (or 4-10 or 5-9).

In refinements, the emulsion has viscosity value in a range of 50 to 100000 cP (or 1000-60000 cP, 2000-40000 cP or 3000-10000 cP), as measured at 25° C.

In refinements, the emulsion has a pH value that remains within 0.1, 0.2, 0.3, 0.5, 0.7, 1, 1.2, or 1.5 pH units of its original pH value (e.g., higher relative to initial value, or lower relative to initial value) upon formation for a period of 10 to 84 days at a storage temperature of 4° C. to 40° C. For example, the emulsion exhibits minimal or low pH change when subjected to an accelerated pH stability test at 10, 28, 56, or 84 days for storage at 4° C., 25° C., or 40° C. as generally described in the examples.

In refinements, the emulsion has an emulsion instability of 15% or less when subjected to a freeze-thaw cycle test, for example an emulsion instability of at least 0.1, 1, 2, 2.5, or 3% and/or up to 4, 6, 8, 10, 12, 15, or 20%. For example, the emulsion exhibits minimal or low instability when subjected to (three) freeze-thaw cycles at −20° C. (freeze)/40° C. (thaw) as generally described in the examples.

In refinements, the emulsion remains stable after formation for a period of 7 days (e.g., at a 20-30° C. or about 25° C. storage temperature) when combined with NaCl at a concentration of 50 mM, 100 mM, 250 mM, or 500 mM in the emulsion. For example, the emulsion remains stable (e.g., no visible separation) when subjected to an ionic strength environmental stress test as generally described in the examples.

In refinements, the emulsion remains stable after formation for a period of 7 days (e.g., at a 20-30° C. or about 25° C. storage temperature) when combined with CaCl₂ at a concentration of 5 mM, 10 mM, 25 mM, or 50 mM in the emulsion. For example, the emulsion remains stable (e.g., no visible separation) when subjected to an ionic strength environmental stress test as generally described in the examples.

In refinements, the emulsion is in the form of a cosmetic composition selected from the group consisting of a lotion (e.g., body/skin lotion, sprayable lotion, sunscreen lotion, hairstyling lotion, natural lotion), a cream (e.g., moisturizing cream, cleansing cream, sunscreen cream, hair care cream, anti-aging cream, lightweight cream, beautifying balm cream), a body milk, and a hydrating serum (e.g., face or skin hydrating serum).

In refinements, the emulsion is in the form of a lotion (e.g., a basic lotion as in the formulation examples); the oil is present in an amount in a range of 8 to 12 wt. % relative to the emulsion; the water is present in an amount in a range of 78 to 86 wt. % relative to the emulsion; the emulsifier composition is present in an amount of 2.5 to 5.5 wt. % relative to the emulsion; and the emulsion further comprises: a pH-adjusting agent, 1.5 to 4.5 wt. % of an active agent (e.g., glycerin), 0.2 to 0.6 wt. % of a thickening agent, and 0.3 to 0.7 wt. % of a preservative.

In refinements, the emulsion is in the form of a sprayable lotion (e.g., as in the formulation examples); the oil is present in an amount in a range of 10 to 20 wt. % relative to the emulsion; the water is present in an amount in a range of 73 to 85 wt. % relative to the emulsion; the emulsifier composition is present in an amount of 0.8 to 4 wt. % relative to the emulsion; and the emulsion further comprises: a pH-adjusting agent, 1.5 to 4.5 wt. % of an active agent (e.g., glycerin), 0.05 to 0.5 wt. % of a thickening agent, and 0.3 to 0.7 wt. % of a preservative.

In refinements, the emulsion is in the form of a body milk (e.g., as in the formulation examples); the oil is present in an amount in a range of 2 to 6 wt. % relative to the emulsion; the water is present in an amount in a range of 90 to 98 wt. % relative to the emulsion; the emulsifier composition is present in an amount of 0.8 to 4 wt. % relative to the emulsion; and the emulsion further comprises: a pH-adjusting agent, 0.5 to 3 wt. % of an active agent (e.g., isopropyl myristate), 0.3 to 0.9 wt. % of a thickening agent, and 0.5 to 2 wt. % of a preservative.

In refinements, the emulsion is in the form of a hydrating serum (e.g., as in the formulation examples); the oil is present in an amount in a range of 2 to 6 wt. % relative to the emulsion; the water is present in an amount in a range of 85 to 95 wt. % relative to the emulsion; the emulsifier composition is present in an amount of 0.8 to 4 wt. % relative to the emulsion; and the emulsion further comprises: a pH-adjusting agent, 1.5 to 4.5 wt. % of an active agent (e.g., glycerin and hyaluronic acid), and 0.5 to 2 wt. % of a preservative.

In refinements, the emulsion is in the form of a cream cleanser (e.g., as in the formulation examples); the oil is present in an amount in a range of 3 to 10 wt. % relative to the emulsion; the water is present in an amount in a range of 76 to 88 wt. % relative to the emulsion; the emulsifier composition is present in an amount of 0.3 to 3 wt. % relative to the emulsion; and the emulsion further comprises: a pH-adjusting agent, 1 to 5 wt. % of an active agent (e.g., glycerin), 0.2 to 0.7 wt. % of a thickening agent, 1 to 8 wt,% of a surfactant, and 0.5 to 2 wt. % of a preservative.

In refinements, the emulsion is in the form of a sunscreen (e.g., as in the formulation examples); the oil is present in an amount in a range of 10 to 40 wt. % relative to the emulsion; the water is present in an amount in a range of 30 to 50 wt. % relative to the emulsion; the emulsifier composition is present in an amount of 0.5 to 5 wt. % relative to the emulsion; and the emulsion further comprises: a pH-adjusting agent, 2 to 8 wt. % of an active agent (e.g., glycerin), 0.05 to 0.5 wt. % of a chelating agent, 0.5 to 2 wt. % of a thickening agent, 1 to 10 wt,% of a surfactant, 0.3 to 3 wt. % of a preservative, and 2 to 40 wt. % of a UV filter.

In refinements, the emulsion is in the form of a moisturizing cream (e.g., as in the formulation examples); the oil is present in an amount in a range of 10 to 22 wt. % relative to the emulsion; the water is present in an amount in a range of 72 to 85 wt. % relative to the emulsion; the emulsifier composition is present in an amount of 0.5 to 8 wt. % relative to the emulsion; and the emulsion further comprises: 1 to 5 wt. % of an active agent (e.g., glycerin), and 0.1 to 0.6 wt. % of a thickening agent.

In refinements, the emulsion is in the form of a lightweight cream (e.g., as in the formulation examples); the oil is present in an amount in a range of 1.5 to 5 wt. % relative to the emulsion; the water is present in an amount in a range of 87 to 96 wt. % relative to the emulsion; the emulsifier composition is present in an amount of 1 to 5 wt. % relative to the emulsion; and the emulsion further comprises: a pH-adjusting agent, 0.5 to 2 wt. % of an active agent, and 0.5 to 2 wt. % of a preservative.

In refinements, the emulsion is in the form of a hairstyling lotion (e.g., as in the formulation examples); the oil is present in an amount in a range of 1.5 to 5 wt. % relative to the emulsion; the water is present in an amount in a range of 88 to 97 wt. % relative to the emulsion; the emulsifier composition is present in an amount of 1 to 5 wt. % relative to the emulsion; and the emulsion further comprises: a pH-adjusting agent, 0.02 to 0.5 wt. % of a thickener, 0.5 to 2 wt. % of an active agent, and 0.5 to 2 wt. % of a preservative.

In refinements, the emulsion is in the form of a beautifying balm (e.g., as in the formulation examples); the oil is present in an amount in a range of 12 to 25 wt. % relative to the emulsion; the water is present in an amount in a range of 55 to 70 wt. % relative to the emulsion; the emulsifier composition is present in an amount of 1.5 to 6 wt. % relative to the emulsion; and the emulsion further comprises: a pH-adjusting agent, 1 to 6 wt. % of a thickener, 3 to 8 wt. % of a preservative, 1 to 6 wt. % of a pigment, and 3 to 8 wt. % of a UV filter.

In refinements, the emulsion is in the form of an anti-aging cream (e.g., as in the formulation examples); the oil is present in an amount in a range of 12 to 25 wt. % relative to the emulsion; the water is present in an amount in a range of 55 to 75 wt. % relative to the emulsion; the emulsifier composition is present in an amount of 3 to 7 wt. % relative to the emulsion; and the emulsion further comprises: a pH-adjusting agent, 0.5 to 4 wt. % of a thickener, and 2 to 12 wt. % of an active agent.

In refinements, the emulsion is in the form of a cream (e.g., as in the formulation examples for an oil-free cream); the oil is present in an amount in a range of 10 to 25 wt. % relative to the emulsion; the water is present in an amount in a range of 69 to 79 wt. % relative to the emulsion; the emulsifier composition is present in an amount of 3 to 8 wt. % relative to the emulsion; and the emulsion further comprises: a pH-adjusting agent, and 0.5 to 2 wt. % of a preservative.

In refinements, the emulsion is in the form of a lotion (e.g., as in the formulation examples for a natural lotion) the oil is present in an amount in a range of 9 to 22 wt. % relative to the emulsion; the water is present in an amount in a range of 68 to 80 wt. % relative to the emulsion; the emulsifier composition is present in an amount of 2 to 7 wt. % relative to the emulsion; and the emulsion further comprises: a pH-adjusting agent, 0.1 to 2 wt. % of a thickener, 0.5 to 2 wt. % of a preservative, and 1 to 5 wt. % of an active agent.

While the disclosed methods, biosolids materials, bioliquids materials, emulsifier compositions, and emulsion compositions are susceptible of embodiments in various forms, specific embodiments of the disclosure are illustrated (and will hereafter be described) with the understanding that the disclosure is intended to be illustrative, and is not intended to limit the claims to the specific embodiments descried and illustrated herein.

### BRIEF DESCRIPTION OF THE DRAWINGS

For a more complete understanding of the disclosure, reference should be made to the following detailed description and accompanying drawings wherein:

FIG. 1 illustrates a polymer or biopolymer complex according to the disclosure.

FIG. 2 illustrates a method for extracting an alginate biomass according to the disclosure.

FIG. 3 illustrates an emulsifier composition and corresponding oil-in-water emulsion using an alginate biomass extract according to the disclosure.

### DETAILED DESCRIPTION

The disclosure relates to methods for extracting an alginate biomass as well as related extract compositions. The alginate biomass feedstock is typically processed using various pre-treatment steps such as size reduction, acidification, rinsing, etc. to provide an alginate-containing biomass pulp. The biomass pulp is then subjected to a multi-step or multi-stage extraction process including a first (alkaline) extraction process or step followed by a second (water or dilution) extraction process. The two extraction steps or stages can be performed in the same or different vessels. The first extraction generally involves contacting the biomass pulp with a base such as a carbonic acid salt (e.g., soda ash or sodium carbonate) to form a biomass dough to convert alginate originally present in the biomass pulp (e.g., in alginic acid form, insoluble alginate salt form, and/or soluble alginate salt form) into a water-soluble alginate salt (e.g., sodium alginate). The second extraction generally involves adding water to the biomass dough in amount sufficient to form a suspension of the extracted biomass with the soluble alginate salt, and then allowing sufficient contact or residence time to partition the suspension into a bioliquids phase and a separate biosolids phase.

The biomass dough typically has a water content in a range of about 40-80 wt. %, a base or carbonic acid salt content in a range of about 5-20 wt. %, and a solids (e.g., biomass solids) content in a range of about 15-55 wt. % based on the biomass dough total mass. The biomass dough has balance between solid (biomass) and liquid (water) content to form a semi-solid mixture of dough-like consistency that can be mixed, blended, kneaded, etc. with sufficient mechanical force, but which does not substantially flow or separate/settle as would a solid/liquid suspension. Water can (but need not) be added along with the carbonic acid salt or other base to obtain a desired consistency/liquid content in the biomass dough. In some cases, the biomass pulp already contains sufficient water and only the base is added for dough formation. In various embodiments, the biomass dough can have a base or carbonic acid salt content of at least 5, 6, 7, 8, 10, or 12 wt. % and/or up to 8, 10, 12, 15, or 20 wt. %, based on the biomass dough total mass. In various embodiments, the biomass dough can have a water content of at least 40, 45, 50, 55, 60, or 65 wt. % and/or up to 50, 55, 60, 65, 70, 75, 80, 85, or 90 wt. %, based on the biomass dough total mass. The foregoing amount ranges for water content can reflect the water content of the biomass pulp without any added water, for example where the biomass pulp as provided by upstream pre-treatment steps has sufficient water for the desired biomass dough consistency.

Alternatively, the foregoing amount ranges for water content can reflect the water content of the biomass pulp with added water, for example where the biomass pulp as provided by upstream pre-treatment steps does not have sufficient water for the desired biomass dough consistency. In various embodiments, the biomass dough can have a solids (or biomass solids) content of at least 5, 7, 10, 12, 15, 17, 20, 25, 30, or 35 wt. % and/or up to 20, 25, 30, 40, 45, 50, or 55 wt. %, based on the biomass dough total mass.

The base can generally include any suitable inorganic acid salt or hydroxide of carbonate (CO₃²⁻) that includes a cation capable of forming a water-soluble form of alginate upon ion exchange of the solubilizing cation with an acidic proton in alginic acid and/or a cation forming a water-insoluble alginate salt. Suitably, the cation of the base can be a monovalent or divalent cation for solubilizing alginate and (optionally) other polysaccharides in the biomass pulp, for example including alkali metals (e.g., sodium, potassium), alkali earth metals (e.g., magnesium, calcium, barium), ammonium, etc. For example, a suitable base for solubilizing alginate in the biomass pulp includes a carbonic acid salt such as sodium carbonate or soda ash (Na₂CO₃), whether in hydrate or anhydrous form. More generally, the base can include one or more inorganic acid salts (e.g., a carbonic acid salt) and hydroxides, for example metal or ammonium salts or hydroxides. For example, an inorganic acid salt base can include an anion such as carbonate, bicarbonate, sulfate, bisulfate, nitrate, phosphate, hydrogen phosphate, dihydrogen phosphate, etc. Similarly, an inorganic acid salt base can include a cation such as an alkali metal cation (e.g., sodium, potassium), an alkaline earth metal cation (e.g., magnesium, calcium, barium), ammonium, etc. A hydroxide base can include an alkali metal hydroxide (e.g., sodium or potassium hydroxide), an alkaline earth metal hydroxide (e.g., magnesium, calcium, or barium hydroxide), ammonium hydroxide, etc.

During the first extraction process, the biomass dough is maintained at a relatively low, mild temperature, for example ambient or room temperature (e.g., about 25° C.). In various embodiments, the biomass dough is maintained at a temperature of at least 5, 10, 15, 20, or 25° C. and/or up to 20, 25, 30, or 35° C. The biomass dough is maintained at the relatively low temperature for a time sufficient to convert alginate originally present in the biomass pulp, for example in alginic acid form, insoluble alginate salt form, and/or soluble alginate salt form, into a water-soluble alginate salt, for example sodium alginate, dissolved in the water portion of the biomass dough. The first extraction process is performed at a low temperature to avoid polymer chain breakdown and molecular weight decrease of the alginate and other polymers in the biomass, in particular to preserve the polymer (or biopolymer) complexes that present in the initial biomass and which provide beneficial properties for use as an emulsifier. In contrast, a typical hot-water extraction at about 60-90° C. or 70-80° C. can efficiently extract alginates and other components from the biomass, but the elevated temperatures also contribute to polymer chain breakdown (e.g., hydrolysis) and polymer complex breakdown during the extraction. It is believed that the lower temperatures of the first extraction can still efficiently extract alginates and other components from the biomass because of the comparatively higher concentration of the carbonic acid salt or other base in the liquid (water) phase resulting from the relatively low-water content dough composition. In contrast, a typical water extraction using a large excess of water forming a flowable biomass suspension (e.g., at least 85, 90, or 95 wt. % water with relatively low solids content) would have a lower concentration of the carbonic acid salt or other base in the liquid (water) phase because of the high water content. The total contact, residence, or extraction time for the first extraction process is not particularly limited, but suitably is about 30-60 minutes, for example at least 5, 10, 15, 20, 30, 40 or 60 min and/or up to 30, 60, 90, 120, 180, or 240 min. During the first extraction, the biomass dough is suitably mixed, agitated, kneaded, etc. to enhance contact between alginate originally present in the biomass pulp in its various forms with the base for conversion to a water-soluble alginate salt. Suitable mixing/contact vessels for such materials with a dough-like consistency are known in the art. The total time for the first extraction is generally sufficient to convert at least about 10, 12, 15, 20, 22, or 25 wt. % and/or up to 20, 25, 30, 35, or 40 wt. %, for example about 20-25 wt. % of the alginate originally present in the biomass pulp to a water-soluble alginate salt. Similarly, at least about 60, 65, 70, 75, or 80 wt. % and/or up to 75, 80, 85, or 90 wt. %, for example about 75-80 wt. % of the alginate originally present in the biomass pulp remains in an (insoluble) alginate form in the solids portion of the biomass pulp after the first extraction (e.g., and in the solids portion of biosolids after the second extraction). The relatively low-temperature extraction process according to the disclosure provides comparable conversion efficiencies for water-soluble alginate salt formation to those of high-temperature extraction processes, but without the corresponding disadvantages of the high-temperature processes (e.g., destruction of the high-molecular weight polymer complexes between alginate and protein materials).

The biomass suspension typically has a water content in a range of about 80-90 wt. % or 80-98 wt. %, and a solids (e.g., biomass solids) content in a range of about 10-20 wt. % or 2-20 wt. % based on the biomass suspension total mass. The water content of the biomass suspension is generally greater than that of the biomass dough, for example being at least 2, 4, 6, 10, 15, or 20 wt. % and/or up to 10, 20, 30, 40 or 50 wt. % higher than the biomass dough water content. Similarly, the solids content of the biomass suspension is generally lower than that of the biomass dough, for example being at least 1, 2, 4, 6, 10, 15, or 20 wt. % and/or up to 10, 20, 30, 40, or 50 wt. % lower than the biomass dough solids content. The additional water added to the biomass dough is sufficient to form a flowable mixture with an aqueous or water liquid continuous phase with dispersed solids suspended therein. In various embodiments, the biomass suspension can have a water content of at least 70, 75, 80, 85, 90, 92, 94, or 96 wt. % and/or up to 85, 90, 95, or 98 wt. %, based on the biomass suspension total mass. In various embodiments, the biomass suspension can have a solids (or biomass solids) content of at least 2, 3, 4, 6, 8, 10, or 12 wt. % and/or up to 4, 7, 10, 12, 15, 20, 25, or 30 wt. %, based on the biomass suspension total mass.

During the second extraction process, the biomass suspension is likewise maintained at a relatively low, mild temperature, for example ambient or room temperature (e.g., about 25° C.) as well as at relatively cold ambient temperature (e.g., down to about 4° C.). In various embodiments, the biomass suspension is maintained at a temperature of at least 4, 5, 10, 15, 20, or 25′C and/or up to 20, 25, 30, or 35° C. Analogous to the first extraction, the second (water or dilution) extraction is performed at a low temperature to avoid polymer chain breakdown and molecular weight decrease of the alginate and other polymers in the biomass. The excess water added during the second extraction provides additional solvent medium (water) and contact time for further increasing total extraction efficiency of the water-soluble alginate salt as well as other components of interest including one or more of proteins, amino acids, and cellulose. By performing the extraction in two separate stages each at relatively mild temperatures, the process obtains a high extraction efficiency or yield, but without the attendant polymer chain and/or polymer complex breakdown and molecular weight decrease associated with a high-temperature extraction typically required for such extraction efficiencies.

During the second extraction, the biomass suspension is partitioned or segregated, typically via centrifugation but without any mixing or agitation, for a time sufficient to form a bioliquids phase and a separate biosolids phase therein. The bioliquids phase includes water and at least a portion of the water-soluble alginate salt extracted from the biomass pulp dissolved therein. The biosolids phase includes proteins, optionally amino acids, cellulose, and at least a portion of the water-soluble alginate salt extracted from the biomass pulp. The total contact, residence, or extraction time for the second extraction process is not particularly limited, but suitably is up to about 24-72 hours, for example at least 1, 2, 3, 4, 6, 12, 18, 24, 36, or 48 hr and/or up to 6, 9, 12, 24, 36, 48, or 72 hr. The second extraction can be performed in the same vessel as that for the first extraction (e.g., but without active mixing, agitation, etc.) or in a different (downstream) vessel. In addition to providing time for settling or segregation, the second extraction can also provide further conversion of biomass alginate to a water-soluble alginate salt (i.e., increased relative to that in the biomass dough). In various embodiments, at least about 10, 12, 15, 20, 22, or 25 wt. % and/or up to 20, 25, 30, 35, or 40 wt. %, for example about 20-25 wt. %, of the alginate originally present in the biomass pulp is recovered as a water-soluble alginate salt in biomass suspension, with at least some water-soluble alginate salt in each of the bioliquids and biosolids phases, for example dissolved or otherwise dispersed in the water or aqueous portion of each phase.

Similarly, at least about 60, 65, 70, 75, or 80 wt. % and/or up to 75, 80, 85, or 90 wt. %, for example about 75-80 wt. %, of the alginate originally present in the biomass pulp remains in an (insoluble) alginate form in the solids portion of the biosolids after the second extraction.

Given the different distribution of components between the two bioliquids and biosolids phases, the two phases typically have different end uses. Accordingly, after completion of the second extraction process, the bioliquids and biosolids phases can be separated using any suitable process, for example via centrifugation, settling, or other gravimetric, mechanical, or physical separation process. The two separated phases can then be used as components for different downstream end products. For example, the biosolids typically contain substantial amounts of proteins and cellulose in addition to some water-soluble alginate, which can make the biosolids particularly suitable for use as an emulsifier, such as in a cosmetic formulation. Suitably, the biosolids are not subjected to an alginate precipitation step (e.g., using calcium chloride, ethanol, etc.), which helps to preserve the biopolymer complex between alginates and proteins in the biosolids. The bioliquids typically contain higher levels of alginate, and thus can be useful when alginate itself is desired end product (e.g., obtainable after a calcium chloride, ethanol, or other precipitation separation from the bioliquids). In other cases, it can be desirable to incorporate the biosolids and bioliquids components into an end product, but at different proportions than those in the partitioned (but unseparated) biomass suspension. For example, a bioleather end product can incorporate both biosolids and bioliquids components. A bioleather according to the disclosure can be in the form of a fabric or textile, for example containing about 10-50 wt. %, 20-50 wt. % or 30-40 wt. % of conventional textile components and about 50-90 wt. %, 50-80 wt. %, or 60-70 wt. % of biosolids and/or bioliquids components combined (e.g., biosolids and/or bioliquids), for example where the conventional textile components and (wet or dried) biosolids/bioliquids sum to 100 wt. %. In embodiments, the (wet or dried) biosolids/bioliquids portion of the bioleather can include at least 50, 60, 70, or 80 wt. % and/or up to 70, 80, 90, 95, or 100 wt. % (wet or dried) bioliquids. In embodiments, the (wet or dried) biosolids/bioliquids portion of the bioleather can include at least 5, 10, 20, or 30 wt. % and/or up to 20, 30, 40, or 50 wt. % (wet or dried) biosolids.

The bioliquids phase typically constitutes about 70-80 wt. %, 50-80 wt. %, or 50-90 wt. % of the biomass suspension. Similarly, the biosolids phase typically constitutes about 20-30 wt. %, 20-50 wt. %, or 10-50 wt. % of the biomass suspension. This partitioning between bioliquids and biosolids represents the combined water/liquid content and solids (e.g., inorganics, organics such as proteins, amino acids, alginates and other polysaccharides, whether in dissolved, suspended, or complexed form in the water), for example before or after phase separation to provide separate bioliquids and biosolids materials, but prior to drying or other water removal steps. At this step, there are generally little or no byproducts of the extraction besides biosolids and bioliquids, such that the bioliquids and biosolids contents sum to at least 95, 98, 99, or 100 wt. %.

In a representative embodiment, the water content for the biosolids ranges from about 92-94 wt. % and the solids content ranges from about 6-8 wt. %. From the solids portion, approximately 57% (or about 50-60%) corresponds to organic matter and 43% (or about 40-50%) to inorganic matter, where approximately 16%-27% (or about 10-30%) of the organic matter is proteins, approximately 56% (or about 50-60%) of the organic matter is cellulose, and the rest is composed of alginates, (non-cellulose) fibers, fatty acids and free amino acids. The biosolids typically have a cellulose content ranging from 40 to 70 wt. % or 50 to 60 wt. % relative to total organic content, for example at least 40, 45, 50, or 55 wt. % and/or up to 55, 60, 65, or 70 wt. %. More generally, the biosolids phase can have a water content in a range of 84 to 96 wt. % relative to the biosolids phase, for example at least 84, 86, 88, 90, or 92 wt. % and/or up to 90, 92, 94, or 96 wt. %. The biosolids phase can have an inorganic content in a range of 2 to 8 wt. % relative to the biosolids phase, for example at least 2, 3, 4, or 5 wt. % and/or up to 4, 5, 6, 7, or 8 wt. %. The biosolids phase can have an organic content in a range of 2 to 8 wt. % relative to the biosolids phase, for example at least 2, 3, 4, or 5 wt. % and/or up to 4, 5, 6, 7, or 8 wt. %. Similarly, the total solid content in the biosolids as a sum of inorganics and organics (e.g., dissolved, suspended, complexed, etc.) can be in a range of 4 to 16 wt. %, for example at least 4, 6, 8, or 10 wt. % and/or up to 8, 10, 12, 14, or 16 wt. %. The foregoing ranges are expressed relative to the biosolids phase on a wet-weight or total basis (i.e., including water/liquid weight therein). The total water, inorganics, and organics contents can sum to at least 95, 98, 99, or 100 wt. %. The inorganics can include cations such as those from the water-soluble alginate salt, whether in dissolved, suspended, or complexed form in the water. The inorganics also can include salts and minerals such as sodium, magnesium, calcium, potassium, iron, and silicon. The organics can include protein, amino acids, alginates, lipids, fibers, cellulose, and/or other polysaccharides, whether in dissolved, suspended, or complexed form in the water.

The distribution of biosolids components can also be expressed on dry weight basis, for example excluding water content, but including all solids whether in dissolved, suspended, or complexed form. In embodiments, the biosolids phase can have an inorganic content in a range of 20 to 80 wt. % on a dry weight basis, for example at least 20, 30, 40, 50, or 60 wt. % and/or up to 40, 50, 60, 70, or 80 wt. %. Similarly, the biosolids phase can have an organic content in a range of 20 to 80 wt. % on a dry weight basis, for example at least 20, 30, 40, 50, or 60 wt. % and/or up to 40, 50, 60, 70, or 80 wt. %.

The biosolids generally have a favorable (total) amino acid profile, which primarily includes amino acids (or amino acid residues) in bound or polymerized or oligomerized form in proteins extracted from the biomass, but which can also include free (monomer) amino acid in unbound form. The biosolids typically have a protein content ranging from 5 to 40 wt. % or 15 to 30 wt. % relative to total organic content, for example at least 5, 7, 10, 12, 15, or 20 wt. % and/or up to 12, 16, 20, 25, 30, 35, or 40 wt. %. In some embodiments, the biosolids also contain at least some free amino acids. While the biosolids can include any of a variety of amino acids, the biosolids suitably include substantial amounts of one or more of glutamic acid, aspartic acid, glycine, leucine, alanine, and arginine. These amino acids are present in particularly high relative amounts in biosolids obtained from sargassum extracts.

These amino acids also exhibit good skin care properties, in particular aspartic acid and alanine for moisturizing and nourishing properties. In addition, the presence of glutamic, arginine, glycine, leucine, lysine, proline, and methionine can have benefits in skin regeneration. The various amino acids can have revitalizing properties that help diminish facial lines and wrinkles, as well as positive effects for strengthening the skin's surface and providing skin protection against harmful substances. Individual amino acids can provide additional or more specific benefits when incorporated into a cosmetic or other skin care composition. For example, glutamic acid can serve as a pH buffer or pH-adjusting agent, and its structure aids in binding water molecules within the skin, therefore moisturizing the skin. Aspartic acid is a non-essential amino acid that is an important building block of collagen and elastin, and that hydrates the skin. Aspartic acid also can be used to set the pH of a cosmetic product (buffering). Glycine improves the skin's elasticity, and it quickly penetrates deep into the inner layers of the skin barrier, down to the dermis, which contains the skin-strengthening protein collagen. Once there, glycine helps stimulate collagen production. This process can help repair damage for healthier, stronger skin. Glycine also works to improve the visible signs of aging; improves moisture retention; increases the production of collagen and strengthens the skin; and promotes skin repair and regeneration.

Alanine can be used to balance moisture levels on the skin, thus providing improved hydration, for example being particularly suited to cosmetic and moisturizing products such as foundations, lotions, creams, and serums. Leucine can help to reduce the appearance of fine lines and wrinkles. Arginine can help to restore visible skin damage, for example being used in cosmetics and skincare products to protect the skin from free radicals, increase the skin's visible hydration levels, and support collagen production. Arginine is a natural moisturizing factor component, a powerful antioxidant, and has the ability to support collagen production. Phenylalanine, tyrosine, and tryptophan can absorb UV light with an absorbance maximum at 280 nm due to the presence of aromatic groups in their structure, although typically only phenylalanine is present from this group in biosolids extracted according to the disclosure (e.g., being free from or without detectable levels of tyrosine and/or tryptophan).

The various favorable properties related to the amino acid distribution of the biosolids are observed when the amino acids present in the form of either or both of proteins and free amino acids. For example, in some embodiments, the total amino acids include both aspartic acid and alanine, and the aspartic acid and the alanine are present in a combined amount in a range of 15 to 25 wt. % or 10 to 30 wt. % relative to the total amino acids, such as at least 10, 12, 15, 17, or 20 wt. % and/or up to 18, 20, 22, 25, or 30 wt. %.

Alternatively or additionally, in some embodiments, aspartic acid, glutamic acid, glycine, alanine, arginine, and/or leucine can be individually present in an amount in a range of 5 to 20 wt. % or 10 to 15 wt. %, such as at least 5, 6, 7, 8, 9, 10, or 12 wt. % and/or up to 8, 10, 12, 14, 16, 18, or 20 wt. %, where each amino acid can be present in a different amount relative ot the other amine acids. In some embodiments, aspartic acid can be the single most abundant amino acid. In some embodiments, glutamic acid can be the single most abundant amino acid. In some embodiments, aspartic acid and glutamic acid combined can be the two most abundant amino acids. Alternatively or additionally, in some embodiments, the total amino acids include glutamic acid, aspartic acid, glycine, leucine, alanine, and arginine; and the glutamic acid, the aspartic acid, the glycine, the leucine, the alanine, and the arginine are present in a combined amount in a range of 40 to 60 wt. % or 30 to 70 wt. % relative to the total amino acids, such as at least 30, 35, 40, 45, or 50 wt. % and/or up to 55, 55, 60, 65, or 70 wt. %. The foregoing ranges for the amino acid profiles are expressed for the total amino acids (i.e., amino acids in bound or free form), but analogous ranges can be expressed for (i) the amino acid (or amino acid residue) profile in the protein portion of the biosolids and/or (ii) the amino acid profile in the free amino acid portion of the biosolids.

The separated biosolids can be further processed after extraction, for example to provide a form suitable for incorporation into one or more end use products. In embodiments, the separated biosolids can be dried, for example using suitable technique such as oven-drying, lyophilization, and freeze-drying. After drying, the biosolids can have a water or moisture content of about 1-20 wt. %, 2-10 wt. %, or 3-7 wt. %. Alternatively or additionally, the biosolids can be treated with one or more divalent crosslinking agents, for example including ionic crosslinkers and/or covalent crosslinkers, in particular enzymatic crosslinkers. Examples of suitable ionic crosslinkers include copper, calcium, barium, zinc, magnesium, and nickel cations (e.g., delivered in salt form). Some examples of suitable end-use products for biosolids (e.g., dried, crosslinked, or otherwise) include a dried emulsifier, thickener or filler agent; a nutritional supplement; fibers such as for use in the textile industry; and strong, crosslinked sheet materials.

In a representative embodiment, the water content of bioliquids is about 98 wt. % or higher, with approximately 2 wt. % solids content. From the solids portion, approximately 50% represents organic matter and the other 50% is inorganic matter, with the organic portion being mostly composed of alginates with a lower percentage of proteins. More generally, the bioliquids phase can have a water content in a range of 90 to 99 wt. % or 90 to 99.5 wt. % relative to the bioliquids phase, for example at least 90, 92, 94, 96, or 98 wt. % and/or up to 92, 95, 97, 98, 99, or 99.5 wt. %. The bioliquids phase can have an inorganic content in a range of 0.2 or 0.5 to 9 wt. % relative to the bioliquids phase, for example at least 0.2, 0.5, 0.7, 1, 1.5, 2, 3, 4, 5, or 6 wt. % and/or up to 3, 4, 5, 6, 7, 8, or 9 wt. %. The bioliquids phase can have an organic content in a range of 0.2 or 0.5 to 9 wt. % relative to the biosolids phase, for example at least 0.2, 0.5, 0.7, 1, 1.5, 2, 3, 4, 5, or 6 wt. % and/or up to 3, 4, 5, 6, 7, 8, or 9 wt. %. Similarly, the total solid content in the bioliquids as a sum of inorganics and organics (e.g., dissolved, suspended, complexed, etc.) can be in a range of 1 to 10 wt. % or 0.5 to 10 wt. %, for example at least 0.5, 1, 1.5, 2, 3, 4, 5, 6, or 7 wt. % and/or up to 2, 3, 4, 5, 6, 7, 8, 9, or 10 wt. %. The foregoing ranges are expressed relative to the bioliquids phase on a wet-weight or total basis (i.e., including water/liquid weight therein).

The total water, inorganics, and organics contents can sum to at least 95, 98, 99, or 100 wt. %. The inorganics and organics in the bioliquids can generally encompass components as described above for the biosolids. However, the bioliquids typically have a higher fraction of the alginate extracted from the original biomass as compared to the biosolids. Further, the bioliquids typically contain little or no cellulose from the original biomass, for example being free from cellulose or containing not more than 0.01, 0.1, or 1 wt. % cellulose relative to either the bioliquids phase as a whole or the organic content thereof.

The distribution of bioliquids components can also be expressed on dry weight basis, for example excluding water content, but including all solids whether in dissolved, suspended, or complexed form. In embodiments, the bioliquids phase can have an inorganic content in a range of 20 to 80 wt. % on a dry weight basis, for example at least 20, 30, 40, 50, or 60 wt. % and/or up to 40, 50, 60, 70, or 80 wt. %. Similarly, the bioliquids phase can have an organic content in a range of 20 to 80 wt. % on a dry weight basis, for example at least 20, 30, 40, 50, or 60 wt. % and/or up to 40, 50, 60, 70, or 80 wt. %.

The bioliquids generally have a relatively high concentration or fraction of water-soluble alginate from the original biomass. In some embodiments, the bioliquids can have a (water-soluble) alginate content ranging from about 0.5-6 wt. %, 1-4 wt. %, or 2-3 wt. % in the liquid phase. Relative to total organic content in the liquid phase of the bioliquids, typically about 97-99.5 wt. %, 98-99 wt. %, or 99-99.5 wt. % is water-soluble alginate and about 0.5-3 wt. %, 1-2 wt. %, or 0.5-1 wt. % is protein.

The separated bioliquids can be further processed after extraction, for example to provide a form suitable for incorporation into one or more end use products. In embodiments, the separated bioliquids can be dried, for example using suitable technique such as oven-drying, lyophilization, and freeze-drying. The dried bioliquid typically contains about 10-20 wt. % water and about 80-90 wt. % solids, where the solids have a similar distribution of inorganic and organic components as above. Alternatively or additionally, the alginates in the bioliquids can be separated or purified, for example using a suitable precipitation technique (e.g., calcium chloride, ethanol, or acid treatment). Alternatively or additionally, the bioliquids can be treated with one or more divalent crosslinking agents, for example including ionic crosslinkers and/or covalent crosslinkers, in particular enzymatic crosslinkers. Examples of suitable ionic crosslinkers include copper, calcium, barium, zinc, magnesium, and nickel cations (e.g., delivered in salt form). Some examples of suitable end-use products for bioliquids (e.g., dried, crosslinked, or otherwise) include films, for example as a polymeric plastic replacement. In some embodiments, a drying technique can be used to treat bioliquids in order to make thin films, which films can then be treated with different divalent crosslinking agents to form strong, malleable and thin materials that could be used as a plastic replacement alternative.

In embodiments, the polymeric components extracted from the original biomass can be present in one or both of the biosolids phase and the bioliquids phase in the form of a polymer (or biopolymer) complex. The polymer complex is generally a network of polymer chains including alginate chains, protein chains, and (in some embodiments) cellulose chains bound together by electrostatic/ionic forces, such as from alginate and corresponding cations, and hydrogen bonding forces, such as from hydroxyl and amino groups in the polymers alginate, protein, and cellulose chains. Thus, the polymer complex generally also includes various cations, such as sodium, magnesium, potassium, iron, calcium, or other metal cations present in the water-soluble alginate salt form. The polymer complex is typically present in both of the biosolids and the bioliquids phases, and the specific composition of the polymer complex in each phase can be the same or different. For example, alginate and proteins are typically extracted into both the biosolids and the bioliquids phases, although a higher fraction of total alginate in the biomass is typically recovered in the bioliquids phase, and the differing relative amounts of alginate and proteins extracted into each phase can be reflected in the polymer complex composition. Similarly, cellulose is typically extracted primarily into the biosolids phase, so the biosolids polymer complex can further include complexed cellulosic chains, whereas the bioliquids polymer complex can be free or substantially free of complexed cellulosic chains.

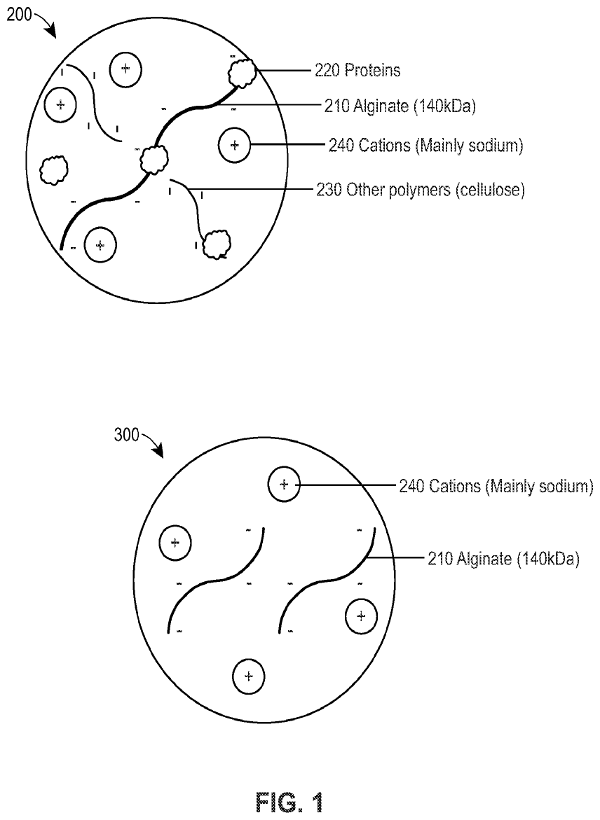

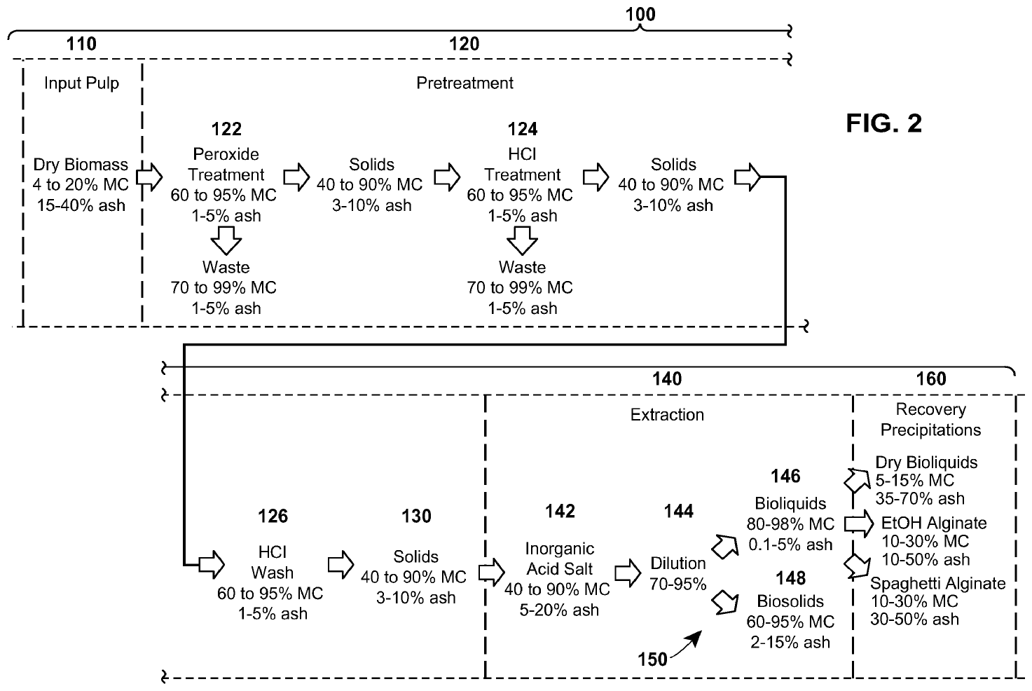

FIG. 1 qualitatively illustrates a polymer or biopolymer complex 200 according to the disclosure. As illustrated, the complex 200 includes alginate chains 210, protein chains 220, and (in some embodiments) cellulosic chains 230 bound together in a high-molecular weight polymeric network via electrostatic/ionic forces and hydrogen bonding forces. As further illustrated, the complex 200 includes cations 240, such as sodium cations, which provide electrostatic/ionic binding interactions with the anionic alginate chains (i.e., including carboxylate or —C(═O)O— groups). As a comparison, FIG. 2 also qualitatively illustrates a typical commercial sodium alginate material 300 including alginate chains 210 and cations 240. Without protein and/or cellulosic chains to provide a network or crosslinks between the alginate chains 210, the alginate material 300 typically has a much lower molecular weight (e.g., corresponding to individual alginate chains vs. complexed alginate, protein, and cellulosic chains). For example, a polymer complex according to the disclosure can have a peak molecular weight or an average molecular weight (e.g., number-average or weight-average) in a range of about 30 kDa to 3000 kDa, 500 kDa to 2000 kDa, or 400 kDa to 3000 kDa, for example at least 20, 30, 40, 50, 70, 100, 150, 200, 250, 300, 400, 500, 600, 700, 800, 900, or 1000 kDa and/or up to 200, 300, 400, 500, 600, 800, 1000, 1200, 1400, 1600, 1800, 2000, 2500, or 3000 kDa. While some peroxide pretreatments can provide relatively lower molecular weight values (e.g., down to about 30 kDa) when using relatively high peroxide amounts, milder peroxide pretreatments can provide relatively higher molecular weight values (e.g., at least about 300, 400, or 500 kDa) when using relatively low peroxide amounts. In contrast, non-complexed alginate chains in a commercial sodium alginate typical have individual alginate chains ranging from about 100-240 kDa. The substantially higher molecular weights of the polymer complex as compared to typical alginate chains reflects a benefit and result of the relatively mild, non-destructive nature of the cold extraction that retains the high molecular weights and the polymer complex structure. The high-molecular weight polymer complexes have particularly useful properties as emulsifiers. In contrast, more aggressive, high-temperature extraction techniques can be efficient at recovering alginate from biomass, but typically destroy the otherwise useful polymer complex in the process.

In embodiments, the extraction method further includes precipitating the water-soluble alginate salt from one or more extraction components, for example including the biomass suspension (i.e., biosolids and bioliquids combined), the biosolids phase (i.e., after separation from bioliquids), and/or the bioliquids phase (i.e., after separation from biosolids).

The precipitation step can include conversion of the water-soluble alginate salt to a water-insoluble alginate salt such as a calcium alginate salt, for example by adding calcium chloride to the biomass suspension or bioliquids, or otherwise performing an ion exchange process to exchange sodium or other cation in the water-insoluble alginate salt with calcium or other cation forming an insoluble salt. Other divalent cations that can be used for precipitation include copper, zinc, and magnesium (e.g., added in a suitable salt form).

Alternatively, the precipitation step can include a solvent-based precipitation, for example by adding ethanol or similar solvent to the biomass suspension or bioliquids.

In embodiments, the alginate-containing biomass (or biomass pulp) that is extracted includes or is exclusively sargassum, for example including one or more sargassum species such as Sargassum fluitans, Sargassum natans, etc. Sargassum fluitans and Sargassum natans are particularly common Caribbean species of the Sargassum genus, but any type of sargassum species from the several hundred known sargassum species and sub-species can be used in the disclosed extraction method to provide corresponding sargassum-based biosolids and bioliquids with similar properties and/or component profiles. More generally, the process can be applied to any type of alginate-containing biomass, for example seaweed or algae (e.g., brown, red, or green), such as kelp, etc. in addition to sargassum. Sargassum is particularly suitable because, although it typically has lower levels of alginate as compared to other seaweeds such as kelp, it typically contains higher levels of proteins and amino acids, in particular having an amino acid profile that is particularly suitable for emulsification, moisturizing of skin, nourishment of skin, etc.

In embodiments, one or more pretreatment steps can be performed on an alginate-containing biomass in order to provide the alginate-containing biomass pulp to be extracted by the disclosed methods. In a first pretreatment step, an alginate-containing biomass can be subjected to drying and/or size-reducing steps, for example drying, milling, etc. to form a powder such as between about 280 and 1000 microns. Drying can be performed at a suitable temperature/time to reduce water content of the biomass to about 15 wt. % or less, such about 0.01-12 wt. % or 1-8 wt. %. The alginate-containing biomass can also be subjected to a peroxide treatment or other decoloring treatment (e.g., UV, bleach, or chloroform treatment), for example after drying and side-reduction. The peroxide treatment can include addition of hydrogen peroxide followed by water wash. The peroxide treatment is used for decoloring (e.g., pigment removal) and removal of other impurities such as arsenic, lipids, and other impurities. The peroxide treatment is typically performed at mild temperatures, for example at about 25° C. or within a range or sub-range of 5-35° C. as described above for the two extraction processes. This peroxide treatment also typically uses relatively low concentrations of hydrogen peroxide, for example about 0.5-15 wt. %, 1-10 wt. %, 3-5 wt. % or 5-8 wt. % hydrogen peroxide relative to the total biomass. This is in contrast conventional hydrogen peroxide treatments, which can use up to about 20 wt. % or more hydrogen peroxide in combination with higher temperatures (e.g., up to 80° C.), more extreme acidic or basic pH ranges (e.g., less than 4, 5, 6, or 7 or higher than 8, 9, 10, or 11), other chemicals (acid and alkali), and/or UV light to assist the treatments, which can in turn further degrade the desirable high molecular weight biopolymer complex that can be recovered in the disclosed methods. In some embodiments, the peroxide treatment according to the disclosure can be performed at higher concentrations to improve the color properties of the final extracted component, for example using 15-50 wt. %, 25-45 wt. %, 20-30 wt. % or 30-40 wt. % hydrogen peroxide relative to the total biomass for the peroxide treatment. In such cases, however, the peroxide treatment and subsequent extraction steps are still performed at mild temperatures, for example at about 25° C. or within a range or sub-range of 5-35° C. as described above, which in turn preserves the desirable high molecular weight biopolymer complex as a recovered extraction product. The alginate-containing biomass can also be subjected to an acidification treatment, for example after the peroxide treatment. The acidification treatment can include addition hydrochloric acid or other strong acid (e.g., sulfuric acid, phosphoric acid, nitric acid) to promote seaweed cell wall hydrolysis, formation of insoluble alginic acid, and removal of phenolic compounds and other polysaccharides. Typical polysaccharides that are removed can include fucoidans and laminarians, for example, whereas polysaccharides such as cellulose are retained (i.e., along with the alginic acid). The acid-treated biomass pulp can then be washed with water to remove excess acid and acid-soluble salts, thereby providing the alginate-containing biomass pulp to be extracted.

In some embodiments, the peroxide treatment can be performed over a wide range of peroxide concentrations, for example to control or tailor one or more properties and/or components of the final biosolids and/or biosolids extract. Examples of extract properties and components that can be adjusted or at least partially controlled via the peroxide treatment can include oxidized cellulose/alginate vs. longer-chain cellulose/alginate, total amino acid content of extract, amino acid distribution in extract, fatty acid content of extract, and fiber content of extract. For example, the peroxide treatment can include contacting or otherwise treating the alginate-containing biomass with a peroxide compound (e.g., hydrogen peroxide) in an amount from 0.5 wt. % to 50 wt. % or 0.5 wt. % to 250 wt. % relative to the alginate-containing biomass, such as an amount of at least 0.5, 1, 2, 3, 4, 5, 6, 10, 15, 20, 25, 30, 40, or 50 wt. % and/or up to 2, 3, 4, 5, 6, 8, 10, 12, 15, 20, 25, 30, 35, 40, 45, 50, 60, 80, 100, 150, 200, or 250 wt. %. Alternatively or additionally, the peroxide treatment can include contacting or otherwise treating the alginate-containing biomass with a peroxide solution (e.g., hydrogen peroxide solution) at a concentration of 0.5 wt. % to 50 wt. % peroxide, such as an amount of at least 0.5, 1, 2, 3, 4, 5, 6, 10, 15, 20, 25, or 30 wt. % and/or up to 2, 3, 4, 5, 6, 8, 10, 12, 15, 20, 25, 30, 35, 40, 45, or 50 wt. % peroxide (e.g., in water).

In embodiments, the extraction method includes adding the carbonic acid salt or other base in solid form to the biomass pulp. For example, pellets or powder of sodium carbonate or other alkaline material as the base can be added to the biomass pulp and mixed together. For example, such pellets can be combined with the biomass pulp in a grinder to mix the salt into the pulp, which in turn dissolves the base into the water component thereof to form the dough.

In embodiments, the first extraction process is performed at a pH in a range of 1.5 to 11, for example at a pH value of at least 1.5, 2, 3, 4, 5, 6, 7, 7.5, 8, 9, or 10 and/or up to 3, 5, 7, 8, 9, 10, or 11. For example, in embodiments including an acidification pretreatment, the pH can drop to a substantially acidic value, for example to pH values as low as about 1.5. After washing of excess acid and addition of the base for the first extraction process, the pH value can increase to substantially neutral or basic values during the first extraction process, for example to or at pH values as high as about 11. In embodiments, the second extraction process is performed at a pH in a range of 8.5 to 10.5, for example at a pH value of at least 8.5, 9, or 9.5 and/or up to 9.5, 10, or 10.5. The pH in either or both extraction processes can be adjusted as desired with the carbonic acid salt or other base/alkaline agent.

The disclosure further relates to emulsifier compositions including a biomass extract or portion thereof according to the disclosure as well as emulsion compositions including the emulsifier compositions, for example oil-in-water cosmetic or other emulsions.

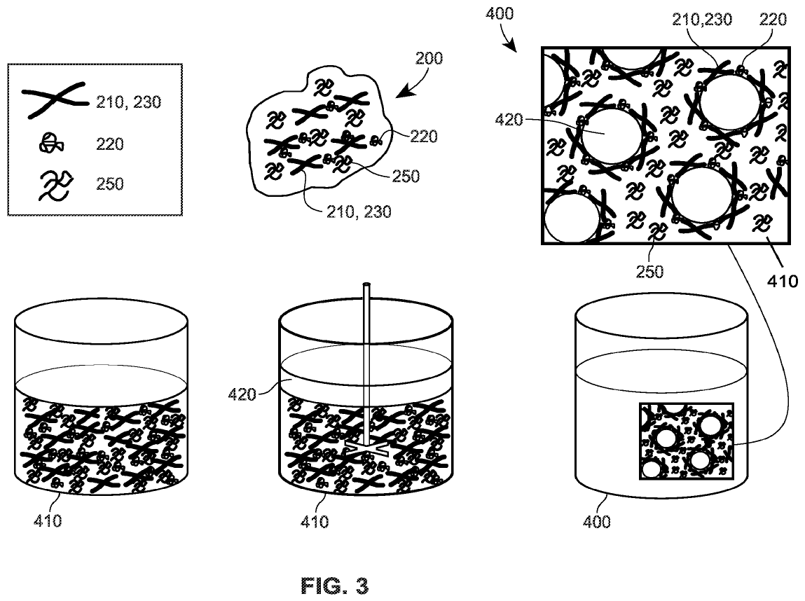

The emulsifier composition generally includes a biosolids alginate biomass extract (or portion thereof) and/or a bioliquids alginate biomass extract (or a portion thereof) in admixture with a thickening agent such as a gum and optionally a preservative. The emulsion composition can include water, oil, and the emulsifier composition. The water can be a primary component of an aqueous phase, such as a continuous aqueous phase. The oil can be a dispersed phase in the continuous aqueous phase. The emulsifier composition can be at an interface between the water/aqueous phase and the oil phase to stabilize the emulsion. For example, as illustrated in FIG. 3, the emulsifier composition can include a thickening agent 250 in combination with one or more biosolids or bioliquids extract components such as the biopolymer complex 200, alginate chains 210, protein chains 220, and/or cellulosic chains 230. The emulsion composition 400 can formed by combining (i) an aqueous phase 410 including water and the emulsifier composition in solution or otherwise (homogeneously) admixed therein with (ii) an oil phase 420, and then mixing or blending the phases 410, 420, for example via any suitable high-shear mixing process or apparatus, to form the emulsion composition 400. As illustrated in the inset to FIG. 4, the emulsion composition 400 can include discrete regions of oil phase 420 in a continuous water 410 medium. In the emulsion composition 400, the thickening agent 250 can be generally distributed throughout the continuous water 410 phase, while the biosolids or bioliquids extract components 200, 210, 220, and/or 230 can be more locally positioned at the oil-water interface.

In embodiments, the biomass extract material component(s) of the emulsifier composition can include one or more of a biosolids alginate biomass extract or portion/fraction thereof and a bioliquids alginate biomass extract or portion/fraction thereof.

The emulsifier composition suitably contains the biomass extract material in an amount of 50 to 98 wt. % or 80 to 90 wt. % relative to the emulsifier composition. More generally, the biomass extract material can be present in an amount of at least 50, 60, 70, 75, 80, 85, 90, 92, 94, or 96 wt. % and/or up to 55, 65, 75, 80, 85, 90, 93, 95, 97, or 98 wt. % relative to the emulsifier composition. The foregoing ranges can apply to the biosolids portion of the biomass extract, the bioliquids portion of the biomass extract, and/or the combined biosolids and bioliquids portions of the biomass extract.

In embodiments, the emulsifier composition suitably contains the thickening agent material (for example a gum) in an amount of 2 to 40 wt. % or 5 to 20 wt. % relative to the emulsifier composition. More generally, the thickening agent can be present in an amount of at least 2, 3, 5, 7, 10, 12, 15, or 20 wt. % and/or up to 6, 8, 10, 12, 15, 17, 20, 25, 30, 35, or 40 wt. % relative to the emulsifier composition. The foregoing ranges can apply a single thickening agent or to the combined amount of all thickening agents in emulsifier composition. Examples of suitable thickening agents include one or more of xanthan gum, guar gum, konjac gum, sclerotium gum, cellulose and cellulose gum, carrageenan, alginates plus crosslinker, crosslinked sargassum bioliquid extracts, carbomers, cetearyl alcohol, glycerol stereate, glycerol stereate citrate, polyacrylates, and hydroxyethylcellulose.

In embodiments, the emulsifier composition suitably contains the preservative in an amount of 1 to 20 wt. % or 2 to 10 wt. % relative to the emulsifier composition. More generally, the preservative can be present in an amount of at least 1, 1.5, 2, 3, 4, 5, or 8 wt. % and/or up to 4, 6, 8, 10, 12, 15, or 20 wt. % relative to the emulsifier composition. The foregoing ranges can apply a single preservative or to the combined amount of all preservatives in emulsifier composition. Examples of suitable preservatives include one or more of pentylene glycol, phenoxyethanol, sodium levulinate, potassium sorbate, honeysuckle extract, caprylhydroxamic acid, sodium anisate, propylene glycol, diazolidinyl urea, iodopropynyl butylcarbamate, and ethanol.

In embodiments, the emulsifier composition suitably has a pH value in a range of 7 to 11 or 8 to 9.5. More generally, the pH value can be at least 7, 7.5, 8, 8.5, 9, 9.5, or 10 and/or up to 8, 8.5, 9, 9.5, 10, 10.5, 11, 11.5, or 12. The pH value of the emulsifier composition can be that which results naturally from its extract, thickening agent, and preservative components in admixture, or it can be adjusted/controlled with a pH-adjusting agent such as an acid, base, or buffer to achieve a desired pH value.

In embodiments, the emulsifier composition suitably has a viscosity value in a range of 200 to 2000 cP or 400 to 1000 cP, as measured in a 10 wt. % aqueous solution of the emulsifier composition at 25° C. More generally, the viscosity of the 10 wt. % aqueous emulsifier solution can be at least 100, 200, 300, 400, 500, or 600 cP and/or up to 300, 500, 700, 1000, 1200, 1500, 2000, 3000, 4000, or 5000 cP. Viscosity values disclosed herein in values, ranges, experimental results, etc. are understood to be measured at 25° C. unless indicated otherwise. The viscosity values can be determined using any suitable viscometer, for example using any of the various viscometers and procedures described in the examples or otherwise.

In embodiments, the emulsion can be in the form of an oil-in-water emulsion.

Alternatively or additionally, the water can be present in an amount in a range of 50 to 98 wt. % relative to the emulsion, for example at least at least 50, 60, 70, 75, 80, 85, 90, 92, 94, or 96 wt. % and/or up to 55, 65, 75, 80, 85, 90, 93, 95, 97, or 98 wt. % relative to the emulsion, such as where the water forms a continuous phase in the emulsion. Alternatively or additionally, the oil can be present in an amount in a range of 2 to 50 wt. % relative to the emulsion, for example at least at least 2, 3, 5, 7, 10, 12, 15, or 20 wt. % and/or up to 6, 8, 10, 12, 15, 20, 25, 30, 35, 40, or 50 wt. % relative to the emulsion, such as where the oil forms a discontinuous or distributed phase in the emulsion. Alternatively or additionally, the emulsifier composition can be present in an amount of 0.2 to 20 wt. % relative to the emulsion, for example at least 0.2, 0.4, 0.6, 0.8, 1, 1.5, 2, 3, 4, 5, or 8 wt. % and/or up to 2, 3, 4, 5, 6, 8, 10, 12, 15, or 20 wt. % relative to the emulsion.

In embodiments, the emulsion can be in the form of a water-in-oil emulsion. For example, the oil can form a continuous phase with a dispersed water/aqueous phase and the emulsifier or components thereof at an interface between the water/aqueous phase and the oil phase to stabilize the emulsion. Alternatively or additionally, the oil can be present in an amount in a range of 50 to 98 wt. % relative to the emulsion, for example at least at least 50, 60, 70, 75, 80, 85, 90, 92, 94, or 96 wt. % and/or up to 55, 65, 75, 80, 85, 90, 93, 95, 97, or 98 wt. % relative to the emulsion, such as where the oil forms a continuous phase in the emulsion. Alternatively or additionally, the water can be present in an amount in a range of 2 to 50 wt. % relative to the emulsion, for example at least at least 2, 3, 5, 7, 10, 12, 15, or 20 wt. % and/or up to 6, 8, 10, 12, 15, 20, 25, 30, 35, 40, or 50 wt. % relative to the emulsion, such as where the water forms a discontinuous or distributed phase in the emulsion.

Alternatively or additionally, the emulsifier composition can be present in an amount of 0.2 to 20 wt. % relative to the emulsion, for example at least 0.2, 0.4, 0.6, 0.8, 1, 1.5, 2, 3, 4, 5, or 8 wt. % and/or up to 2, 3, 4, 5, 6, 8, 10, 12, 15, or 20 wt. % relative to the emulsion.

In embodiments, a ratio of emulsifier composition to oil (or total oil) present in the emulsion is in a range of 1:1.5 to 1:10, 1:2 to 1:8, 1:3 to 1:7, or 1:4 to 1:6 on a weight/weight basis. More generally, the emulsifier composition:oil ratio can be at least 1:1.1, 1:1.2, 1:1.3, 1:1.5, 1:1.7, 1:2, 1:2.5, 1:3, 1:4, 1:5, 1:6, or 1:7 and/or up to 1:1.25, 1:1.4,1:1.6, 1:1.8, 1:2, 1:2.5, 1:3, 1:3.5, 1:4, 1:5, 1:6, 1:7, 1:8, 1:9, 1:10, 1:12, 1:15. Similar ratios and ranges from the foregoing can alternatively apply to a ratio of alginate biomass extract (e.g., biosolids and/or bioliquids) to oil (or total oil) in the emulsion. These ratios reflect an excess of oil relative to emulsifier composition or the biomass extract portion thereof.

The oil is not particularly limited and can generally include any suitable cosmetically acceptable oils or emollients as well as non-cosmetic oils. Examples of suitable oils or emollients include one or more of caprylic/capric triglyceride, coco caprylate caprate, mineral oil, octyldodecanol, coconut oil, grapeseed oil, sunflower oil, squalane, silicones (e.g., dimethicone, silicone quarternium-3, trideceth-12, amodimethicone, c11-15 alketh-12, c11-15 alketh-7, cyclopentasiloxane), avocado oil, olive oil, jojoba oil, almond oil, sunflower wax, beeswax, shea butter, ethers (e.g., dicaprylyl ether), esters (e.g., cetyl esters, ethyl palmate, isopropyl palmitate, octyl pamitate, jojoba esters, octyldodecyl oleate), and limnanthes alba (meadowfoam) seed oil.

In embodiments, the oil can have a polarity index of 15 mN/m or less, between 15-30 mN/m or 15-35 mN/m, or at least 35 mN/m. More generally, the polarity index can be at least 2, 5, 10, 12, 15, 18, 20, 22, 25, 27, 30, 32, 35, 37, 40, 45, or 50 mN/m and/or up to 10, 12, 15, 18, 20, 22, 25, 27, 30, 32, 35, 37, 40, 45, 50, 60, 70, or 80 mN/m. The polarity index for an oil is the interfacial tension of the oil with respect to water. In general, the higher the oil polarity index, the harder it is to emulsify, or properly mix the ingredients together. Oils having a polarity index above 35 mN/m are considered nonpolar, while those having a polarity index below 35 mN are referred to as polar (e.g., medium polar: between 15-30 mN/m or 15-35 mN/m, and highly polar below 15 mN/m).

In embodiments, the emulsion can further include one or more optional components or additives such as preservatives, thickening agents, chelating agents, anti-oxidants, active agents, surfactants, pigments, colorants, UV filters, and/or pH-adjusting agents (e.g., acids, bases, buffers). When present, the additional preservatives and/or thickening agents in the emulsion can be selected from the same options described above for the emulsifier composition components, but the preservatives and/or thickening agents included as a separate emulsion component can be the same of different from those originally in the emulsifier composition. Examples of suitable chelating agents and anti-oxidants include: ethylenediaminetetraacetic acid (EDTA) and tocopherols. Examples of suitable active agents include: glycerin, isopropyl myristate, hyaluronic acid, glycol distearate, panthenol, niacinamide, ceramide, tetrahexyldecyl ascorbate, caprylhydroxamic acid, glyceryl caprylate, chamomile extract, and propanediol. Examples of suitable surfactants/coemulsifiers include: cocamidopropyl betaine, sodium myreth sulfate, decyl glucoside, hydroxysultaine, lauroyl sarcosinate, polyvinylpyrrolidone, sodium cocoyl isethionate, and PEG-100 stearate. Examples of suitable pigments, pearls (or pearlescent pigments), and colorants include: mineral, organic, pearls, titanium dioxide, and iron oxides.

Examples of suitable pH modifiers or buffers include: lactic acid, citric acid, and sodium hydroxide. Examples of suitable UV filters include: titanium dioxide and zinc oxide.

The optional components can be included in any suitable amount, for example 0.01 to 20 wt. % collectively for all optional components relative to the emulsion, such as at least 0.01, 0.1, 0.2, 0.5, 0.8, 1, 2, 3, 5, 7, or 10 wt. % and/or up to 1, 2, 4, 6, 8, 10, 12, 15, or 20 wt. %. Alternatively or additionally, individual optional components can be included in amounts from 0.01 to 5 wt. % or 0.01 to 10 wt. % relative to the emulsion, such as at least 0.01, 0.1, 0.2, 0.3, 0.4, 0.5, 0.6, 0.7, 0, 8, 1, 2, 3, 4, 5, 6, or 8 wt. % and/or up to 0.3, 0.5, 0.7, 1, 1.5, 2, 3, 4, 5, 6, 7, 8, 9, or 10 wt. %. Alternatively or additionally, the combined amount of water, oil, and emulsifier composition can be at least 80, 85, 90, 92, 95, 97, 98, or 99 wt. % and/or up to 85, 88, 90, 92, 94, 96, 98, 99, 99.5, 99.8 wt. % of the emulsion.

In embodiments, the emulsion composition suitably has a pH value in a range of 3 to 11, 4-10, or 5 to 9. More generally, the pH value can be at least 3, 3.5, 4, 4.5, 5, 5.5, 6, 6.5, 7, 7.5, 8, 8.5, 9, 9.5, or 10 and/or up to 4, 4.5, 5, 5.5, 6, 6.5, 7, 7.5, 8, 8.5, 9, 9.5, 10, 10.5, 11, 11.5, or 12. The pH value of the emulsion composition can be that which results naturally from its emulsifier, water, and oil components in admixture, or it can be adjusted/controlled with a pH-adjusting agent such as an acid, base, or buffer to achieve a desired pH value.

In embodiments, the emulsion composition suitably has a viscosity value in a range of 50 to 100000 cP, 1000-60000 cP, 2000-40000 cP, or 3000-10000 cP as measured at 25° C. More generally, the viscosity of the emulsion can be at least 50, 100, 200, 500, 700, 1000, 1200, 1500, 2000, 2500, 3000, 4000, 5000, 6000, 7000, 8000,10000,15000, 20000, or 30000 cP and/or up to 2000, 4000, 6000, 8000, 10000, 15000, 20000, 30000, 40000, 50000, 60000, 80000, or 100000 cP. Viscosity values disclosed herein in values, ranges, experimental results, etc. are understood to be measured at 25° C. unless indicated otherwise. The viscosity values can be determined using any suitable viscometer, for example using any of the various viscometers and procedures described in the examples or otherwise.

### EXAMPLES

The following examples illustrate the methods and compositions disclosed herein, but are not intended to be limiting.

### Example 1—Extraction of Sargassum Biomass and Extract Characterization

### Extraction Process

Sargassum biomass was extracted according to the disclosure to provide biosolids and bioliquids materials. FIG. 2 illustrates the extraction process 100 along with representative material balance information for the various process stages. Sargassum as an alginate-containing biomass was ground and dried for about 10 hours at about 75° C. to form a dried alginate-containing biomass powder 110 with a moisture content (MC) of about 4-12 wt. %. The biomass powder 110 was then subjected to a series of pretreatment steps 120 to provide a corresponding alginate-containing biomass pulp 130 for extraction. The biomass powder 110 was subjected to a peroxide treatment 122 to remove non-target compounds such as pigments and lipids using 3% hydrogen peroxide for about 4 hours at about 25° C. A water rinse was then performed to remove excess hydrogen peroxide and other impurities. The biomass powder 110 was then subjected to an acidification treatment 124 to form water-insoluble alginic acid using 10% hydrochloric acid for about 1 hour at about 25° C. The acidic biomass powder 110 was then washed/rinsed 126 with deionized water for about 1 hour at about 25° C. to remove excess hydrochloric acid, thereby forming the biomass pulp 130.

The biomass pulp 130 was then extracted 140 using a first alkaline extraction process 142 followed by a second water extraction process 144. In the first extraction 142, solid sodium carbonate pellets were added to the biomass pulp 130 and blended in a meat grinder to intimately mix the sodium carbonate and the pulp 130, thereby forming biomass dough. In some instances, water was also added to the pulp 130 in the first extraction 142 in order to provide a suitable dough consistency for the mixture. Insoluble alginic acid initially present in the pulp 130 was solubilized by the sodium carbonate (i.e., as dissolved in the free water portion of the dough). The biomass dough was mixed for about 30-60 minutes at about 25° C. to complete the first extraction 142. In the second extraction 144, additional water was added to the biomass dough in sufficient amounts to create a biomass suspension. The biomass suspension was allowed to sit/settle for about 24-72 hours at about 25° C., thereby partitioning the suspension into a bioliquids phase 146 and a separate biosolids phase 148. The bioliquids phase 146 and the biosolids phase 148 were then separated 150 via centrifugation. The bioliquids phase 146 was then dried as a representative downstream recovery or precipitation process 160. As illustrated, the bioliquids phase 146 could be alternatively processed to form an ethanol-precipitated alginate, a spaghetti alginate, etc. In a typical extraction, the water content for the biosolids 148 ranged from about 92-94 wt. % and the solids content ranged from about 6-8 wt. %.

From the solids portion, approximately 57% corresponded to organic matter and 43% to inorganic matter, where approximately 16% of the organic matter was proteins and the rest was composed of alginates, fibers, fatty acids and free amino acids. Similarly, in a typical extraction, the water content of bioliquids was about 98 wt. % or higher (i.e., as extracted; not yet dried), with approximately 2 wt. % solids content. From the solids portion, approximately 50% represented organic matter and the other 50% was inorganic matter, with the organic portion being mostly composed of alginates with a small percentage of proteins.

### Biosolids and Bioliquids Compositions

Samples of the biosolids extract and the dried bioliquids extract were analyzed for total protein and total amino acid distribution (i.e., including bound amino acids in the proteins and any free amino acids collectively). The protein content of the biosolids was about 16-27 wt. % relative to total organic content in the biosolids. The protein content of the dried bioliquids was about 0.5-1 wt. % relative to total organic content in the bioliquids. The total amino acid distributions for the biosolids and the dried bioliquids are shown in Table 1 below, where the weight-percent entries are for amount of a single amino acid relative to total amino acids on a w/w basis.

TABLE 1
Total Amino Acid Distribution

| Amino Acid | Biosolids | Dried Bioliquids |
|---|---|---|
| Aspartic Acid | 13.1% | 15.7% |
| Threonine | 5.3% | 7.8% |
| Serine | 5.7% | 6.2% |
| Glutamic Acid | 13.6% | 16.1% |
| Proline | 4.3% | 3.9% |
| Glycine | 8.4% | 7.9% |
| Alanine | 7.4% | 7.1% |
| Valine | 6.0% | 6.0% |
| Isoleucine | 4.8% | 4.3% |
| Leucine | 8.1% | 5.4% |
| Tyrosine | 4.2% | 3.9% |
| Phenylalanine | 5.4% | 4.1% |
| Lysine | 4.3% | 3.4% |
| Histidine | 0.0% | 0.0% |
| Arginine | 7.0% | 5.1% |
| Cystine | 0.0% | 0.0% |
| Methionine | 2.4% | 3.0% |

### Example 2—Extraction of Sargassum Biomass and Effect of Pretreatment

Sargassum biomass was extracted as generally described in Example 1 to provide biosolids and bioliquids materials, but with a varying amount of hydrogen peroxide used in the peroxide pretreatment step for decoloring the biomass. For extraction A (“high peroxide”), a solution hydrogen peroxide was used in an amount sufficient to provide 250 wt. % hydrogen peroxide relative to total biomass, resulting in bioliquid and biosolid extracts with a green coloration. A solution hydrogen peroxide was used for extraction B (“low peroxide”) in an amount sufficient to provide 15 wt. % hydrogen peroxide relative to total biomass, resulting in brown biosolids and bioliquids. The organic profile (total proteins, free and bound amino acids, sugars, fibers, and fatty acids) and molecular weight were determined for the biosolids and bioliquids extracts obtained with the high and low peroxide extraction processes.

For the total organic profile analysis, a sample size of 200 grams of the different extracts was used. Total protein was measured by the Dumas method (N×6.25). To determine the amino acid profile, total amino acids (i.e., bound amino acids forming proteins+free amino acids) and free amino acids were measured by high-performance liquid chromatography. The analysis of total, saturated, and unsaturated fatty acids was done using the modified AOAC Method No. 996.06. Gas liquid chromatography was used to determine the sugar profile, while the AOAC International Method No. 991.43 was used to analyze total fibers. The amount of solids and the organic and inorganic percentages were determined using the total solids/volatile solids analysis.

The biosolid extracts were high in moisture (93-95%) with a dry matter range of 5-6.5%. The organic composition of the biosolid phase of the sargassum extract was constituted mainly by fibers (55-75% DW), carbohydrates (12-15% DW), and proteins (5-15% DW). The fiber portion was mostly composed of oxidized celluloses and the carbohydrate portion was mostly composed of oxidized alginates, in the case of the high peroxide treated extraction. For the low peroxide treated extractions, longer chained polymers of celluloses and alginates are expected to be part of the fiber and carbohydrate portions of the sargassum extract, respectively. Fatty acids were found in the sargassum extracts at a low percentage (0.02-0.06%) and sugars were not detected under the conditions tested. Since amino acids in their free form were not detected, it is believed that they are complexed or otherwise attached to the alginates and celluloses forming a bio polymeric complex in the extracts.

Total SolidsNolatile Solids (TSNS) Analysis: The results (n=90) from the TSNS analysis show that there is no significant impact on the TSNS results for the different peroxide treatments in the corresponding extraction products. The results presented are based on extraction processes using a 10-fold dilution of the alkali cake/pulp. Therefore, in an alternative process, the dry matter of the biosolids and bioliquids could be customized and selected as desired depending on the dilution factor used during the process. The amount of total solids present in the biosolids (based on a 10-fold dilution of the alkali cake/pulp) was approximately 5% and it was independent of the hydrogen peroxide treatment. The distribution of organic and inorganic contents within the total solids of the biosolids was about 50-60 wt. % organic solids and about 40-50 wt. % inorganic solids, relative to total solids. The dry matter concentration in the bioliquids (based on a 10-fold dilution of the alkali cake/pulp) was approximately 2% and it was independent of the hydrogen peroxide treatment. The distribution of organic and inorganic contents within the total solids of the bioliquids was about 50-60 wt. % organic solids and about 40-50 wt. % inorganic solids, relative to total solids. Table 2 below summarizes the results for the different extract phases and peroxide treatment concentrations. In Table 2, moisture (water) and total solids are weight percent values relative to the extract (i.e., high or low peroxide treatment), while organic and ash (inorganic) solids are weight percent values relative to the solids portion of the extract.

TABLE 2
Extract Moisture and Solids Distribution

| | Moisture | Total | Organics | Ash |
|---|---|---|---|---|
| Sample | (%) | Solids (%) | (%) | (%) |
| High | 95.19 ± 0.90 | 4.81 ± 0.90 | 54.70 ± 3.45 | 45.30 ± 3.45 |
| Peroxide | | | | |
| Biosolids | | | | |
| High | 98.03 ± 0.006 | 1.97 ± 0.006 | 54.44 ± 0.11 | 45.56 ± 0.11 |
| Peroxide | | | | |
| Bioliquids | | | | |
| Low | 93.50 ± 0.01 | 6.5 ± 0.01 | 56.30 ± 0.03 | 43.70 ± 0.03 |
| Peroxide | | | | |
| Biosolids | | | | |
| Low | 98.2 ± 0.003 | 1.8 ± 0.003 | 51.90 ± 0.07 | 48.10 ± 0.07 |
| Peroxide | | | | |
| Bioliquids | | | | |

Protein Results for Biosolids: The total amount of proteins presents in the high peroxide treated wet biosolids (at a 10-fold dilution of the alkali cake/pulp) was approximately 0.4%, which corresponds to approximately 4 milligrams of protein for each gram of high peroxide treated wet biosolids (at a 10-fold dilution of the alkali cake/pulp). The total amount of proteins presents in the low peroxide treated wet biosolids (at a 10-fold dilution of the alkali cake/pulp) was 0.8%, which corresponds to approximately 8 milligrams of protein for each gram of low peroxide treated wet biosolids (at a 10-fold dilution of the alkali cake/pulp). As the concentration of hydrogen peroxide used to decolor sargassum increases, the more the proteins become denatured. As illustrated in these results, the total protein content in biosolids decreased by 50% when the pulp was treated with a high peroxide level for decoloration as compared to a low peroxide pretreatment.

Total Amino Acid Profile for Biosolids: The results show the total value of amino acids present in the biosolids, including bound amino acids in the form of proteins and free (monomer) amino acids. Free amino acids were not detected under the conditions tested for either high or low peroxide treated biosolids. Table 3 shows the results of the total amino acid content percentage of the wet biosolids (at a 10-fold dilution of the alkali cake/pulp) treated with high hydrogen peroxide. The values for tyrosine, cystine, methionine, tryptophan and histidine are below the detection point therefore they are not shown. Similarly, Table 4 shows the results of the total amino acid content percentage of the wet biosolids (at a 10-fold dilution of the alkali cake/pulp) treated with low hydrogen peroxide. The values for cystine, tryptophan, and histidine are below the detection point therefore they are not shown.

TABLE 3
Amino Acid Content in High Peroxide Treated Wet Biosolids
(at a 10-fold dilution of the alkali cake/pulp)

| | Amino Acid |
|---|---|
| Amino Acid | Distribution (%) |
| Aspartic acid | 13.9% |
| Glutamic acid | 12.7% |
| Leucine | 9.2% |
| Arginine | 8.5% |
| Glycine | 8.5% |
| Alanine | 8.1% |
| Valine | 6.4% |
| Serine | 6.1% |
| Phenylalanine | 6.0% |
| Threonine | 5.8% |
| Proline | 5.7% |
| Isoleucine | 5.1% |
| Lysine | 4.0% |

TABLE 4
Amino Acid Content in 3Low Peroxide Treated Wet Biosolids
(at a 10-fold dilution of the alkali cake/pulp)

| | Amino Acid |
|---|---|
| Amino Acid | Distribution (%) |
| Glutamic acid | 14.5% |
| Aspartic acid | 12.9% |
| Glycine | 9.9% |
| Alanine | 7.9% |
| Leucine | 7.2% |
| Arginine | 7.0% |
| Serine | 5.6% |
| Valine | 5.5% |
| Phenylalanine | 5.2% |
| Proline | 5.1% |
| Threonine | 5.0% |
| Isoleucine | 4.3% |
| Lysine | 4.0% |
| Tyrosine | 3.6% |
| Methionine | 2.1% |

As shown in Tables 3 and 4, the concentrations of individual amino decreased (e.g., by about 40-60, or approximately 50% decrease) when the pulp was treated with high hydrogen peroxide for decoloration as compared to a low peroxide pretreatment.

Additionally, the results show a different distribution of specific amino acids depending on the peroxide pretreatment concentration. As shown in Table 3, the top five most abundant amino acids (i.e., relative to all amino acids present) with the highest concentrations in high peroxide treated biosolids are (in descending order): aspartic acid, glutamic acid, leucine, arginine, and glycine. As shown in Table 4, the top five most abundant amino acids (i.e., relative to all amino acids present) with the highest concentrations in low peroxide treated biosolids are (in descending order): glutamic acid, aspartic acid, glycine, alanine, and leucine.

These results illustrate the ability of the disclosed extraction process to provide extracts that not only have amino acids particularly useful for skin care, but also with specific amino acids and/or an amino acid distribution tailored to a particular skin care use. For example, aspartic acid and alanine are present in a high level relative to other amino acids, and they have moisturizing and nourishing properties ideal for skin care. In addition, the presence of glutamic, arginine, glycine, leucine, lysine, proline, and methionine have potential benefits in skin regeneration. These amino acids have revitalizing properties that help diminish facial lines and wrinkles. They also have positive effects on strengthening the skin's surface and on skin protection against harmful substances.

Fatty Acid Content for Biosolids: The only fatty acids observed were saturated fatty acids with a concentration of 0.02% and 0.06% for high and low peroxide treated wet biosolids (at a 10-fold dilution of the alkali cake/pulp), respectively. Unsaturated, monosaturated, polyunsaturated and trans fatty acids were not detected under the conditions tested.

Fiber Content for Biosolids: There are approximately 35-40 milligrams of fiber for each gram of high peroxide treated wet biosolids (at a 10-fold dilution of the alkali cake/pulp). In the case of low peroxide treated wet biosolids (at a 10-fold dilution of the alkali cake/pulp), there are approximately 40-45 milligrams of fiber for each gram of wet sargassum extract.

Sugar Content for Biosolids: No sugars were detected under the conditions tested.

Bioliquids Analysis: The organic content of the bioliquid portion of the sargassum extract typically only includes carbohydrates (e.g., about 50-70 wt. % or 50-100 wt. % relative to total organic content) such as alginate and/or cellulose, and/or fibers (about 30-50 wt. % relative to total organic content), depending on the hydrogen peroxide treatment, dilution factor, and the separation technique applied. The biopolymer complex present in the bioliquids can be free of cellulose depending on the separation technique. No fatty acids, sugars or proteins were detected in the bioliquids extract under the conditions tested.

Fiber Content for Bioliquids: There are approximately 7-10 milligrams of fiber for each gram of high peroxide treated bioliquids (at a 10-fold dilution of the alkali cake/pulp). In the case of low peroxide treated bioliquids (at a 10-fold dilution of the alkali cake/pulp), there are approximately 8-11 milligrams of fiber for each gram of liquid sargassum extract.

Alginate Content for Bioliquids. The alginate concentration in bioliquids can range from 0.009 g/ml to 0.03 g/ml depending on the hydrogen peroxide treatment, and dilution factor.

Molecular Weight Analysis: Molecular weight analysis of bioliquids treated with high and low peroxide as well as precipitated alginic acid and alginate of non-decolored sargassum extracts was performed using high performance gel permeation chromatography to characterize the molecular weight distribution. Table 5 shows the results of solution polymers in the different samples.

TABLE 5
Retention time (RT) and molecular weight (MW) of the solution
polymers in samples of precipitated alginic acid from
non-peroxide treated bioliquid, precipitated alginate
from non-peroxide treated bioliquid, low-peroxide treated
bioliquid, and high-peroxide treated bioliquid

| | | | | | AVERAGE |
|---|---|---|---|---|---|
| | RT1 | RT2 | MW1 | MW2 | MW |
| SAMPLE | (min) | (min) | (Da) | (Da) | (Da) |
| Non-peroxide | 14.16 | 16.075 | 101,215 | 121,390 | 111,302.5 |
| Treated | | | | | |
| Alginic Acid, | | | | | |
| peak 1 | | | | | |
| Non-peroxide | 17.12 | 18.202 | 16,706 | 15,962 | 16,334 |
| Treated | | | | | |
| Alginic Acid, | | | | | |
| peak 2 | | | | | |
| Non-peroxide | 19.87 | 20.75 | 1,386 | 1,393 | 1,389.5 |
| Treated | | | | | |
| Alginic Acid, | | | | | |
| peak 3 | | | | | |
| Non-peroxide | 13.93 | 13.863 | 992,701 | 1,034,383 | 1,013,542 |
| Treated | | | | | |
| Alginate, | | | | | |
| peak 1 | | | | | |
| Non-peroxide | 20.682 | 20.703 | 1,470 | 1,464 | 1,467 |
| Treated | | | | | |
| Alginate, | | | | | |
| peak 2 | | | | | |
| Non-peroxide | N/A | N/A | N/A | N/A | N/A |
| Treated | | | | | |
| Alginate, | | | | | |
| peak 3 | | | | | |
| Low-peroxide | 23.05 | 22.55 | 109,926 | 154,917 | 132,421.5 |
| Treated | | | | | |
| Bioliquids, | | | | | |
| peak 1 | | | | | |
| Low-peroxide | 28.491 | 28.495 | 2,628 | 2,621 | 2,624.5 |
| Treated | | | | | |
| Bioliquids, | | | | | |
| peak 2 | | | | | |
| Low-peroxide | N/A | N/A | N/A | N/A | N/A |
| Treated | | | | | |
| Bioliquids, | | | | | |
| peak 3 | | | | | |
| High-peroxide | 24.6 | 24.55 | 37,949 | 39,274 | 38,611.5 |
| Treated | | | | | |
| Bioliquids, | | | | | |
| peak 1 | | | | | |
| High-peroxide | N/A | N/A | N/A | N/A | N/A |
| Treated | | | | | |
| Bioliquids, | | | | | |
| peak 2 | | | | | |
| High-peroxide | N/A | N/A | N/A | N/A | N/A |
| Treated | | | | | |
| Bioliquids, | | | | | |
| peak 3 | | | | | |

### Example 3—Emulsifier Compositions with Different Extract Phases

The extraction process according to the disclosure provides extracts with various polymeric and other components from the original alginate-containing biomass such as sargassum. Two different extracts are obtained from the final separation step of the extraction process: a bioliquid and biosolid phase. The bioliquid phase contains most of the alginates extracted from the original biomass while the biosolid phase contains mostly celluloses with some alginates and proteins present as well. This example illustrates the ability of the various extract components to serve as an emulsifier, for instance for an oil-in-water emulsion typical of common cosmetic formulations such as for skin care or other topical application.

In some representative emulsifier compositions, sargassum-extracted bioliquids as well as the alginates precipitated from the bioliquids were used to form emulsifiers. Successful oil-in-water emulsions could be formed with the precipitated alginates, although the emulsion stability was limited by the pH of the solution: The alginate-based emulsifier from the bioliquid phase of the sargassum extracts was most efficient at high pH levels (above 7).

In some other representative emulsifier compositions, sargassum-extracted biosolids were used to form emulsifiers. When the biosolid phase was used as a base for a cosmetic emulsifier, more stable emulsions were obtained, even at pH levels as low as 3.5.

Biosolid extracts treated under different extraction conditions were evaluated as potential emulsifiers. All the different treated extracts were successful at stabilizing emulsions made at various pH levels and oil concentrations. The emulsions prepared had different oil concentrations, sargassum extracts treated under different conditions; either alginates precipitated from bioliquids in their dried or wet state or biosolids; various sargassum extracts concentrations; and several pH levels.

The following oil-in-water emulsions were made with sargassum-extracted bioliquids components or biosolids components under different conditions: (A) 20% sunflower oil-in-water emulsion made with 2% dried alginate precipitated from bioliquids as the emulsifier (pH value above 7); (B) 20% sunflower oil-in-water emulsion made with 10% alginate precipitated from bioliquids as the emulsifier with the alginate suspended in ethanol (pH value above 7); (C) 40% sunflower oil-in-water emulsion made with 20% alginate precipitated from bioliquids as the emulsifier with the alginate suspended in ethanol (pH value above 7); (D) 20% sunflower oil-in-water emulsion made with 2% biosolids as the emulsifier (pH value of 7); (E) 20% sunflower oil-in-water emulsion made with 2% biosolids as the emulsifier (pH value of 4.5); (F) 20% sunflower oil-in-water emulsion made with 2% biosolids as the emulsifier (pH value of 5.8); (G) 10% sunflower oil-in-water emulsion made with 2% biosolids as the emulsifier (pH value of 7); and (H) 10% sunflower oil-in-water emulsion made with 2% biosolids as the emulsifier (pH value of 5).

### Example 4—Emulsifier Compositions with Different Peroxide Pretreatment

Since the emulsions in Example 3 formed with biosolids as the emulsifier resulted in the most stable emulsions, additional biosolids-based emulsifiers obtained from sargassum extracts were evaluated. Three different treated sargassum-extracted biosolids were evaluated using a different peroxide pretreatements: (1) non-decolored biosolids (i.e., no peroxide pretreatment), (2) minimally decolored biosolids (low peroxide treated), and (3) fully decolored (high peroxide treated) biosolids.

The biosolid extracts are high in moisture (93-95%) with a dry matter range of 5-6.5%. The organic composition of the biosolid phase of our sargassum extract is constituted mainly by fibers (55-75% DW), carbohydrates (12-15% DW), and proteins (5-15% DW).

The fiber portion is mostly composed of oxidized celluloses and the carbohydrate portion is mostly composed of oxidized alginates, in the case of the high peroxide treated extraction.

For the low peroxide treated extractions, longer-chained polymers of celluloses and alginates are expected to be part of the fiber and carbohydrate portions of the sargassum extract, respectively. Fatty acids are found in the sargassum extracts at a low percentage (0.02-0.06%) and sugars are not detected under the conditions tested. Since amino acids in their free form were not detected, it is believed that they are attached to the alginates and celluloses forming a biopolymeric complex. The biopolymeric complex also includes various cations, such as sodium, calcium, magnesium, and other metal cations present in the water-soluble form of alginate.

Emulsions made with non-decolored biosolids were very stable. The emulsion finish had a very dark brown color, and a brown pigmentation or residue was transferred to the skin when applied.

The addition of a peroxide pretreatment removed some of the pigments present in the sargassum biomass. The initial peroxide pretreatment consisted of incubating the sargassum biomass in a solution containing low hydrogen peroxide. This small concentration of hydrogen peroxide decolored the pulp. The low-H₂O₂ treated biosolids produced emulsions with a cream-colored finish that did not leave a residue on the skin.

Representative emulsions made with low-H₂O₂ treated biosolids included (A) 25% sunflower oil-in-water emulsion made with 3.5% biosolids, 0.5% xanthan gum, 0.8% pentylene glycol, and water (balance) at pH 7; and (B) 40% sunflower oil-in-water emulsion made with 5.5% biosolids, 0.5% xanthan gum, 0.8% pentylene glycol, and water (balance) at pH 6.66.

A higher concentration peroxide pretreatment was performed in the attempt to make white-colored emulsions. Sargassum biomass treated with high hydrogen peroxide yielded bioliquid and biosolid extracts with a green pigmentation. White emulsions were achieved when using the high-H₂O₂ treated biosolids as an emulsifying raw material.

### Example 5—Emulsifier Compositions with Sargassum Biosolids

In initial testing, low-H₂O₂ treated biosolids extracted from sargassum were used as emulsifiers and were found to have emulsifying properties. Based on the typical moisture content resulting from the extraction process in the biosolids, a thickening agent was added to improve the emulsifying system's rheology and stability

The emulsifier composition of Example 5 included a blend of decolored sargassum biosolid extract (85% w/w; but could be about 80-95% w/w more generally) as an emulsifier, xanthan gum (10% w/w; but could be about 3-10% w/w more generally) as a thickening agent, and pentylene glycol (5% w/w; but could be about 1-5% w/w more generally) as a preservative. The emulsifier composition of Example 5 had a dry matter content of about 15-20 wt. % with a basic pH ranging from about 8.0 to 9.5 (i.e., exhibiting some natural variation for sargassum extracts). The emulsifier composition was a pale green paste, and had a viscosity ranging from about 500 to 900 mPa*s when dissolved in water at a 10% (w/w) concentration and measured at 25° C.

The organic composition of the biosolids is comprised of up to 75% DW cellulose. Depending on the peroxide treatment, cellulose could have no modifications to the structure, yielding a high molecular weight polymer; or it could be present as oxidized cellulose with a shorter-chained polymeric structure. Cellulose is an amphiphilic polymer that can adsorb to the oil-water interface of the emulsion's dispersed phase. When cellulosic molecules self-assemble due to electrostatic interactions at the oil-water interface to form a coating around the oil droplets, they act as a mechanical barrier towards the coalescence of the oil droplets. Depending on the concentration of cellulose present in the emulsion system, the excess of cellulosic molecules, that are not coating the oil droplets, occupy some well-defined physical space in the aqueous phase of the emulsion. With gradually increasing cellulose concentration in the continuous media, the overall system's viscosity increases and changes into one exhibiting solid-like rheological behavior. The resulting stability of the system is improved by limiting the movement of oil droplets within the viscoelastic network of cellulosic molecules in the continuous phase.

When high-molecular-weight polymers are used, the concentration needed to achieve the system's stability is lower than with shorter-chained polymers, as in the case of oxidized cellulose. This was clearly observed in the sargassum biosolid extracts where the efficacy of the biosolids to stabilize an emulsion's system decreases as a more aggressive hydrogen peroxide treatment is applied in the extraction process. Non-hydrogen-peroxide and lower peroxide treated biosolids contain cellulose with higher polymeric chains. High-molecular weight polymers are more efficient emulsifiers since they not only self-assemble at the oil-water interface providing a mechanical barrier to the coalescence of oil droplets, but the adsorbed polymers also provide a repulsive force driven by the entropic penalty when polymer segments from different droplets start to entangle. In the case of high peroxide treated biosolids, these strong steric repulsions driven by the entropic penalty of polymer entanglement are hindered due to the resulting shorter-chained polymer. Oxidized alginate and proteins are also present in the biosolids in significant smaller amounts compared to oxidized cellulose. The complexes of this polysaccharide and proteins with cellulose creates a synergistic effect that improve the stability of emulsions. This cellulose-alginate-protein bio complex could be created mainly by physical adsorption interactions, such as hydrogen bonding and electrostatic attraction interaction.

The sargassum biosolid extracts according to the disclosure show emulsifying stability properties at their original matter state containing a high-water content of up to 95%. The emulsifying efficacy of the biopolymeric complex present in the biosolids is directly proportional to the concentration of the bio complex. Representative emulsifier compositions according to the disclosure are highly efficient emulsifier based on natural, environmentally friendly components a blend formulation containing up to 90% (w/w) sargassum biosolid extract, a thickener, and a preservative.

The thickening agent was added to improve the system's rheology and stability. A low concentration of non-adsorbing hydrocolloids, such as gum Arabic, xanthan gum, guar gum, galactomannans, modified starches, alginates, carrageenans, hyaluronan, and chitosan among others, can have an inhibiting effect on phase separation through the generation of gel-like viscoelastic network of the continuous aqueous phase. Xanthan gum was found to be a particularly suitable thickening agent to enhance the gel-like viscoelastic network of the continuous aqueous phase. Due to the high moisture content of the sargassum biosolid extracts and the shorter-chained cellulosic bio complex, the strong steric repulsion arising from the thickening agents provided a natural, seaweed-based emulsifier with high stability against environmental stresses such as pH, ionic strength, and temperature.

The stability and durability of emulsions depend on the formulation, the device used for emulsification, and operation conditions. In the case of the disclosed sargassum-based emulsifier, the formation of fine and monodisperse oil droplets can be suitably achieved by high-shear force and ultrasonication devices. The formation of fine droplets in conjunction with the adsorbed oxidized cellulose-alginate-protein bio complex on the oil droplets and the viscoelastic network formed by the thickening agents create a robust and stable emulsion system.

### Example 6—Oil-in-Water Emulsion Compositions with Sargassum Emulsifier

Example 6 evaluates the emulsifier composition of Example 5 in many different oil-in-water emulsions representative of typical cosmetic formulations. Example 6 evaluates emulsion stability using the Example 5 emulsifier composition in combination with polar (low, medium, and high polarity) and non-polar emollients, at different emollient concentrations, at different pH levels, at various ionic strength environments, and at several temperatures.

Example 6 also demonstrates the compatibility of the Example 5 emulsifier composition with many different ingredients also commonly used as additives, adjuvants, or other components of cosmetic compositions (e.g., thickeners, surfactants, active ingredients, silicones, waxes, and butters).

Since the polarity of cosmetic oils or emollients can affect the stability and solubility of the emulsion, a diversity of emollients (polar and nonpolar) were evaluated to demonstrate the capability of an emulsifier composition according to the disclosure to keep an emulsion stable. The polarity index for an oil is the interfacial tension of the oil with respect to water.

In general, the higher the oil polarity index, the harder it is to emulsify, or properly mix the ingredients together. Oils having a polarity index above 35 mN/m are considered nonpolar, while those having a polarity index below 35 mN are referred to as polar (e.g., medium polar: between 15-30 mN/m or 15-35 mN/m, and highly polar below 15 mN/m).

As illustrated further below, the Example 5 emulsifier composition typically performs better with polar emollients. A lesser amount of the emulsifier composition is needed to maintain stable emulsions made with polar oils. Nonetheless, the Example 5 emulsifier composition is still capable of maintaining stable emulsions with nonpolar emollients with a slightly higher amount of emulsifier composition in the formula. The following emollients have been used to make stable emulsions using the the Example 5 emulsifier composition: squalene (nonpolar, 46.2 mN/m), mineral oil (nonpolar, 43.7 mN/m), coconut oil (nonpolar), dimethicone (polar, 26.6 mN/m), cyclopentasiloxane, coco-caprylate/caprate (medium polar, 24.8 mN/m), octyldodecanol (medium polarity, 24.8 mN/m), octyl palmitate (23.1 mN/m), dicaprylyl ether (22.1-30.9 mN/m), isopropyl palmitate (28.8 mN/m), caprylic/capric triglyceride (polar, 21.3 mN/m), sunflower oil (polar, 19.3 mN/m), avocado oil (polar, 18.3 mN/m), olive oil (polar, 16.9 mN/m), grapeseed oil (polar), blend of avocado, jojoba, and almond oil (polar, 18.3, 20.8, and 20.3 mN/m, respectively), sunflower wax, and shea Butter. The physical stability of emulsions made with the Example 5 emulsifier composition was assessed by several methods, including accelerated aging and freeze-thaw cycling.

Emulsion Stability—Accelerated Aging: The accelerated aging test determines the influence of different emollients, emollient concentrations, and emulsifier composition concentrations on the pH of emulsions over time. This is a stability test for emulsions made with the Example 5 emulsifier composition where accelerating aging was attained by increasing the temperature during incubation or storage. Three different incubation temperature variables were tested: 4° C., 25° C., and 40° C. The total incubation time was 12 weeks. Visual observations, pH measurements, and viscosity measurements are recorded at 0, 4, 8 and 12 weeks.

The efficacy of the Example 5 emulsifier composition to stabilize emulsions made with polar and nonpolar emollients at different concentrations was determined by inducing accelerated aging in the emulsions. The emollients used for the study included: squalane, mineral oil, coconut oil, coco caprylate caprate, octyldodecanol, caprylic/capric triglyceride, sunflower oil, avocado oil, olive oil, grapeseed oil, and a blend of jojoba, almond and olive oil. Emulsions were made with oil concentrations ranging from 8.5% up to 40%. Also, the stability of Example 5 emulsifier composition was studied under different pH conditions, making emulsions with pH as low as 3.5 and as high as 9.

The oil-in-water emulsion formulations used for this example contained four ingredients: water, the Example 5 emulsifier composition, emollient, and preservative. The amount of emulsifier composition used depended on the emollient used and its concentration. For example, at a 10% squalane concentration, 3-5% emulsifier composition was used; at a 25% squalane concentration, 3.5 and 4% emulsifier composition was used; and at a 40% squalane concentration, 5.5% emulsifier composition was used. For the squalane, coco caprylate/caprate, octyldodecanol, and mineral oil, the more challenging concentration was 10%. All the other concentrations behaved the same as with the other emollients. The amount of emulsifier composition used was also slightly increased when working at pH values of 3.5 and 4.

All of the oil-in-water emulsions prepared for the example remained stable after the 12-week period. The emulsions looked unchanged visually with no apparent phase separation. Some formulations were discarded due to microbial contamination. Regarding pH measurements, no significant pH differences were observed across the different formulations and at different incubation temperatures during the length of the study, except for the formulations at high pH (pH=9). Tables 6-13 show the pH stability results for squalane, caprylic/capric triglyceride, coco caprylate caprate, sunflower oil, grapeseed oil, mineral oil, octyldodecanol, and natural blend composed of avocado oil (40% w/w), almond oil (30% w/w), and jojoba oil (30% w/w). The tables indicate the amount of Example 5 emulsifier composition (denoted “EC”) along with the amount of emollient (denoted “Oil”) for each test. In general, basic formulations incubated at 4° C. showed a 10% decrease in pH after the first 4 weeks with no significant change beyond week 4. For basic formulations incubated at 25° C. and 4000, a decrease in pH of up to 25% was observed during the week 4 with no further change in pH after that time.

TABLE 6
Accelerated pH Stability Results for Squalene

| Squalane | Temp (° C.) | Day 0 | Day 10 | Day 28 | Day 56 | Day 84 |
|---|---|---|---|---|---|---|
| EC 3.5% \| Oil 25% | 4 (° C.) | 9.17 | | 8.33 | 8.42 | — |
| | 25 (° C.) | 9.17 | | 6.75 | | — |
| | 40 (° C.) | 9.17 | | 7.60 | 7.65 | |
| | 50 (° C.) | 9.17 | 7.92 | | | |
| EC 3.5% \| Oil 25% | 4 (° C.) | 5.22 | | 5.31 | 5.32 | 5.32 |
| | 25 (° C.) | 5.22 | | 6.35 | 6.80 | 6.91 |
| | 40 (° C.) | 5.22 | | 5.42 | 6.51 | 5.59 |
| | 50 (° C.) | 5.22 | 5.23 | | | |
| EC 4% \| Oil 25% | 4 (° C.) | 4.39 | | 4.02 | 4.41 | 4.53 |
| | 25 (° C.) | 4.39 | | 4.87 | 5.29 | 5.45 |
| | 40 (° C.) | 4.39 | | 4.20 | 4.58 | 5.09 |
| | 50 (° C.) | 4.39 | 4.51 | | | |
| EC 4% \| Oil 25% | 4 (° C.) | 3.58 | | 3.67 | 3.71 | 3.70 |
| | 25 (° C.) | 3.58 | | 3.83 | — | — |
| | 40 (° C.) | 3.58 | | 3.53 | 3.59 | 3.88 |
| | 50 (° C.) | 3.58 | 3.64 | | | |
| EC 3% \| Oil 10% | 4 (° C.) | 5.03 | | 5.06 | 5.11 | 5.00 |
| | 25 (° C.) | 5.03 | | 6.25 | 4.75 | 5.55 |
| | 40 (° C.) | 5.03 | | 5.32 | — | — |
| | 50 (° C.) | 5.03 | 4.83 | | | |
| EC 4% \| Oil 10% | 4 (° C.) | 7.18 | | 6.77 | 6.75 | 6.77 |
| | 25 (° C.) | 7.18 | | 6.73 | 5.57 | — |
| | 40 (° C.) | 7.18 | | 7.07 | — | — |
| | 50 (° C.) | 7.18 | 6.80 | | | |
| EC 5% \| Oil 10% | 4 (° C.) | 7.04 | | 7.58 | 7.53 | 7.48 |
| | 25 (° C.) | 7.04 | | 6.94 | 6.97 | — |
| | 40 (° C.) | 7.04 | | 6.99 | 5.83 | — |
| | 50 (° C.) | 7.04 | 6.85 | | | |
| EC 5.5% \| Oil 40% | 4 (° C.) | 7.56 | | 7.35 | 7.68 | 7.45 |
| | 25 (° C.) | 7.56 | | 7.22 | — | 5.68 |
| | 40 (° C.) | 7.56 | | 7.05 | 6.94 | 5.94 |
| | 50 (° C.) | 7.56 | 6.76 | | | |

TABLE 7
Accelerated pH Stability Results for Caprylic/Capric Triglyceride

| Caprylic/Capric | | | | | | |
|---|---|---|---|---|---|---|
| Triglyceride | Temp (° C.) | Day 0 | Day 10 | Day 28 | Day 56 | Day 84 |
| EC 3.5% \| Oil 25% | 4 (° C.) | 9.23 | | 8.42 | 8.42 | 7.67 |
| | 25 (° C.) | 9.23 | | 6.91 | 6.70 | 6.53 |
| | 40 (° C.) | 9.23 | | 7.65 | 7.75 | 6.79 |
| | 50 (° C.) | 9.23 | 7.53 | | | |
| EC 3.5% \| Oil 25% | 4 (° C.) | 6.23 | | 6.22 | 6.34 | 6.54 |
| | 25 (° C.) | 6.23 | | 5.67 | 5.80 | 5.50 |
| | 40 (° C.) | 6.23 | | 5.52 | 5.37 | 5.52 |
| | 50 (° C.) | 6.23 | 5.59 | | | |
| EC 4% \| Oil 25% | 4 (° C.) | 4.41 | | 4.47 | 4.46 | 4.47 |
| | 25 (° C.) | 4.41 | | 4.52 | 4.61 | 4.57 |
| | 40 (° C.) | 4.41 | | 4.28 | 4.29 | 4.14 |
| | 50 (° C.) | 4.41 | 4.38 | | | |
| EC 4% \| Oil 25% | 4 (° C.) | 3.56 | | 3.74 | 3.67 | 3.60 |
| | 25 (° C.) | 3.56 | | 3.74 | 3.75 | 3.69 |
| | 40 (° C.) | 3.56 | | 3.62 | 3.51 | — |
| | 50 (° C.) | 3.56 | 3.66 | | | |
| EC 3% \| Oil 10% | 4 (° C.) | 7.12 | | 6.83 | 6.85 | 5.95 |
| | 25 (° C.) | 7.12 | | 5.87 | 5.85 | 5.72 |
| | 40 (° C.) | 7.12 | | 6.27 | 6.09 | 6.17 |
| | 50 (° C.) | 7.12 | 5.72 | | | |
| EC 5.5% \| Oil 40% | 4 (° C.) | 7.54 | | 7.78 | 7.71 | 7.47 |
| | 25 (° C.) | 7.54 | | 6.94 | 5.76 | 5.45 |
| | 40 (° C.) | 7.54 | | 7.09 | 6.81 | 6.58 |
| | 50 (° C.) | 7.54 | 6.94 | | | |

TABLE 8
Accelerated pH Stability Results for Coco Caprylate/Caprate

| Coco Caprylate | | | | | | |
|---|---|---|---|---|---|---|
| Caprate | Temp (° C.) | Day 0 | Day 10 | Day 28 | Day 56 | Day 84 |
| EC 3.5% \| Oil 25% | 4 (° C.) | 9.15 | | 8.52 | 8.29 | 8.10 |
| | 25 (° C.) | 9.15 | | 6.78 | 6.62 | 5.98 |
| | 40 (° C.) | 9.15 | | 7.77 | 7.77 | — |
| | 50 (° C.) | 9.15 | 7.49 | | | |
| EC 3.5% \| Oil 25% | 4 (° C.) | 6.76 | | 7.07 | 6.77 | 6.81 |
| | 25 (° C.) | 6.76 | | 6.57 | 6.12 | 6.19 |
| | 40 (° C.) | 6.76 | | 6.32 | 6.36 | 6.56 |
| | 50 (° C.) | 6.76 | 6.14 | | | |
| EC 4% \| Oil 25% | 4 (° C.) | 3.94 | | 4.04 | 4.03 | 4.33 |
| | 25 (° C.) | 3.94 | | 4.08 | 4.22 | CONT |
| | 40 (° C.) | 3.94 | | 3.88 | 3.86 | 3.86 |
| | 50 (° C.) | 3.94 | 3.75 | | | |
| EC 4% \| Oil 25% | 4 (° C.) | 3.71 | | 3.78 | 3.67 | 3.88 |
| | 25 (° C.) | 3.71 | | 3.81 | 4.01 | CONT |
| | 40 (° C.) | 3.71 | | 3.71 | 3.74 | 3.97 |
| | 50 (° C.) | 3.63 | 3.60 | | | |
| EC 4% \| Oil 10% | 4 (° C.) | 7.18 | | 6.74 | 6.74 | 6.86 |
| | 25 (° C.) | 7.18 | | 6.47 | 5.77 | — |
| | 40 (° C.) | 7.18 | | 6.02 | 6.15 | 6.50 |
| | 50 (° C.) | 7.18 | 5.99 | | | |
| EC 5% \| Oil 10% | 4 (° C.) | 7.04 | | 7.59 | 6.99 | 7.03 |
| | 25 (° C.) | 7.04 | | 6.49 | 6.44 | 5.99 |
| | 40 (° C.) | 7.04 | | 7.03 | 6.63 | 6.36 |
| | 50 (° C.) | 7.04 | 6.92 | | | |
| EC 5.5% \| Oil 40% | 4 (° C.) | 7.58 | | 7.59 | 6.99 | 7.03 |
| | 25 (° C.) | 7.58 | | 6.49 | 6.44 | 5.99 |
| | 40 (° C.) | 7.58 | | 7.03 | 6.63 | 6.36 |
| | 50 (° C.) | 7.58 | 3.59 | | | |

TABLE 9
Accelerated pH Stability Results for Sunflower Oil

| Sunflower Oil | Temp (° C.) | Day 0 | Day 10 | Day 28 | Day 56 | Day 84 |
|---|---|---|---|---|---|---|
| EC 3.5% \| Oil 25% | 4 (° C.) | 8.99 | | 8.66 | 8.43 | 8.24 |
| | 25 (° C.) | 8.99 | | 7.15 | 6.45 | 6.13 |
| | 40 (° C.) | 8.99 | | 7.64 | 7.19 | 6.73 |
| | 50 (° C.) | 8.99 | 7.60 | | | |
| EC 3.5% \| Oil 25% | 4 (° C.) | 7.30 | | 7.41 | 7.05 | 7.14 |
| | 25 (° C.) | 7.30 | | 6.42 | 6.35 | 6.32 |
| | 40 (° C.) | 7.30 | | 6.35 | 5.97 | 5.76 |
| | 50 (° C.) | 7.30 | 6.38 | | | |
| EC 4% \| Oil 25% | 4 (° C.) | 4.14 | | 4.13 | 4.14 | 4.52 |
| | 25 (° C.) | 4.14 | | 4.31 | 4.64 | — |
| | 40 (° C.) | 4.14 | | 4.15 | 4.07 | 4.11 |
| | 50 (° C.) | 4.14 | 4.09 | | | |
| EC 4% \| Oil 25% | 4 (° C.) | 3.73 | | 3.75 | 3.84 | 3.86 |
| | 25 (° C.) | 3.73 | | 3.81 | 4.06 | — |
| | 40 (° C.) | 3.73 | | 3.73 | 3.76 | 3.88 |
| | 50 (° C.) | 3.73 | 3.75 | | | |
| EC 3% \| Oil 10% | 4 (° C.) | 6.43 | | 6.36 | 6.26 | 6.52 |
| | 25 (° C.) | 6.43 | | 6.26 | 6.31 | 6.44 |
| | 40 (° C.) | 6.43 | | 6.75 | 6.74 | 6.65 |
| | 50 (° C.) | 6.43 | 5.46 | | | |
| EC 5.5% \| Oil 40% | 4 (° C.) | 6.42 | | 6.56 | 6.98 | 6.76 |
| | 25 (° C.) | 6.42 | | 6.43 | 5.70 | 5.80 |
| | 40 (° C.) | 6.42 | | 5.96 | 5.65 | 5.52 |
| | 50 (° C.) | 6.42 | 5.66 | | | |

TABLE 10
Accelerated pH Stability Results for Grape Seed Oil

| Grape Seed Oil | Temp (° C.) | Day 0 | Day 10 | Day 28 | Day 56 | Day 84 |
|---|---|---|---|---|---|---|
| EC 3.5% \| Oil 25% | 4 (° C.) | 9.05 | | 8.52 | 8.49 | 8.22 |
| | 25 (° C.) | 9.05 | | 6.44 | 6.68 | 6.80 |
| | 40 (° C.) | 9.05 | | 7.71 | 7.37 | 6.11 |
| | 50 (° C.) | 9.05 | 7.24 | | | |
| EC 3.5% \| Oil 25% | 4 (° C.) | 7.26 | | 7.36 | 7.66 | 6.86 |
| | 25 (° C.) | 7.26 | | 6.91 | 6.68 | 5.90 |
| | 40 (° C.) | 7.26 | | 6.89 | 6.78 | 6.09 |
| | 50 (° C.) | 7.26 | 5.83 | | | |
| EC 4% \| Oil 25% | 4 (° C.) | 3.92 | | 4.06 | 4.08 | 4.19 |
| | 25 (° C.) | 3.92 | | 4.05 | CONT | — |
| | 40 (° C.) | 3.92 | | 3.89 | 4.57 | 4.33 |
| | 50 (° C.) | 3.92 | 4.10 | | | |
| EC 4% \| Oil 25% | 4 (° C.) | 3.71 | | 3.91 | 3.99 | 3.95 |
| | 25 (° C.) | 3.71 | | 3.82 | 3.98 | — |
| | 40 (° C.) | 3.71 | | 4.03 | 4.10 | 4.18 |
| | 50 (° C.) | 3.71 | 3.74 | | | |
| EC 4% \| Oil 10% | 4 (° C.) | 7.35 | | 7.37 | 7.65 | 6.81 |
| | 25 (° C.) | 7.35 | | 5.98 | 6.48 | 5.73 |
| | 40 (° C.) | 7.35 | | 6.57 | 6.53 | 6.23 |
| | 50 (° C.) | 7.35 | 5.68 | | | |
| EC 5% \| Oil 10% | 4 (° C.) | 7.85 | | 7.73 | 7.78 | 7.20 |
| | 25 (° C.) | 7.85 | | 5.76 | 5.98 | 5.58 |
| | 40 (° C.) | 7.85 | | 7.09 | 5.85 | 5.02 |
| | 50 (° C.) | 7.85 | 6.42 | | | |
| EC 5.5% \| Oil 40% | 4 (° C.) | 7.34 | | 7.60 | 7.63 | 7.66 |
| | 25 (° C.) | 7.34 | | 6.99 | 7.13 | — |
| | 40 (° C.) | 7.34 | | 7.06 | 5.82 | 6.85 |
| | 50 (° C.) | 7.34 | 6.22 | | | |

TABLE 11
Accelerated pH Stability Results for Mineral Oil

| Mineral Oil | Temp (° C.) | Day 0 | Day 10 | Day 28 | Day 56 | Day 84 |
|---|---|---|---|---|---|---|
| EC 3.5% \| Oil 25% | 4 (° C.) | 9.27 | | 8.52 | 8.49 | 8.22 |
| | 25 (° C.) | 9.27 | | 6.44 | 6.68 | 6.80 |
| | 40 (° C.) | 9.27 | | 7.71 | 7.37 | 6.11 |
| | 50 (° C.) | 9.27 | 7.56 | | | |
| EC 3.5% \| Oil 25% | 4 (° C.) | 7.64 | | 7.36 | 7.66 | 6.86 |
| | 25 (° C.) | 7.64 | | 6.91 | 6.68 | 5.90 |
| | 40 (° C.) | 7.64 | | 6.89 | 6.78 | 6.09 |
| | 50 (° C.) | 7.64 | 6.86 | | | |
| EC 4% \| Oil 25% | 4 (° C.) | 4.17 | | 4.06 | 4.08 | 4.19 |
| | 25 (° C.) | 4.17 | | 4.05 | CONT | — |
| | 40 (° C.) | 4.17 | | 3.89 | 4.57 | 4.33 |
| | 50 (° C.) | 4.17 | 4.01 | | | |
| EC 4% \| Oil 25% | 4 (° C.) | 3.81 | | 3.91 | 3.99 | 3.95 |
| | 25 (° C.) | 3.81 | | 3.82 | 3.98 | — |
| | 40 (° C.) | 3.81 | | 4.03 | 4.10 | 4.18 |
| | 50 (° C.) | 3.81 | 3.74 | | | |
| EC 4% \| Oil 10% | 4 (° C.) | 7.12 | | 7.37 | 7.65 | 6.81 |
| | 25 (° C.) | 7.12 | | 5.98 | 6.48 | 5.73 |
| | 40 (° C.) | 7.12 | | 6.57 | 6.53 | 6.23 |
| | 50 (° C.) | 7.12 | 6.66 | | | |
| EC 5% \| Oil 10% | 4 (° C.) | 7.40 | | 7.73 | 7.78 | 7.20 |
| | 25 (° C.) | 7.40 | | 5.76 | 5.98 | 5.58 |
| | 40 (° C.) | 7.40 | | 7.09 | 5.85 | 5.02 |
| | 50 (° C.) | 7.40 | 7.07 | | | |
| EC 5.5% \| Oil 40% | 4 (° C.) | 7.84 | | 7.60 | 7.63 | 7.66 |
| | 25 (° C.) | 7.84 | | 6.99 | 7.13 | — |
| | 40 (° C.) | 7.84 | | 7.06 | 5.82 | 6.85 |
| | 50 (° C.) | 7.84 | 7.18 | | | |

TABLE 12
Accelerated pH Stability Results for Octyldodecanol

| Octyldodecanol | Temp (° C.) | Day 0 | Day 10 | Day 28 | Day 56 | Day 84 |
|---|---|---|---|---|---|---|
| EC 3.5% \| Oil 25% | 4 (° C.) | 9.23 | | 8.64 | 8.40 | 8.36 |
| | 25 (° C.) | 9.23 | | 7.27 | PS | — |
| | 40 (° C.) | 9.23 | | 7.75 | 7.34 | 6.46 |
| | 50 (° C.) | 9.23 | 7.36 | | | |
| EC 3.5% \| Oil 25% | 4 (° C.) | 7.14 | | 7.39 | 7.05 | 7.1 |
| | 40 (° C.) | 7.14 | | 6.77 | 6.86 | 6.40 |
| | 50 (° C.) | 7.14 | 6.75 | | | |
| EC 4% \| Oil 25% | 4 (° C.) | 4.02 | | 4.22 | 4.24 | 4.68 |
| | 25 (° C.) | 4.02 | | 4.26 | — | 4.50 |
| | 50 (° C.) | 4.02 | 3.98 | | | |
| EC 4% \| Oil 25% | 4 (° C.) | 3.76 | | 3.91 | 3.99 | 4.06 |
| | 25 (° C.) | 3.76 | | 3.82 | 4.21 | — |
| | 40 (° C.) | 3.76 | | 4.03 | 4.14 | 4.39 |
| | 50 (° C.) | 3.76 | 3.89 | | | |
| EC 4% \| Oil 10% | 4 (° C.) | 6.35 | | 6.77 | 7.00 | 7.09 |
| | 25 (° C.) | 6.35 | | 6.57 | 6.59 | 6.54 |
| | 40 (° C.) | 6.35 | | 6.63 | 6.75 | — |
| | 50 (° C.) | 6.35 | 5.79 | | | |
| EC 5% \| Oil 10% | 4 (° C.) | 7.31 | | 7.75 | 7.56 | 7.49 |
| | 25 (° C.) | 7.31 | | 6.76 | 6.75 | 6.01 |
| | 40 (° C.) | 7.31 | | 6.91 | 5.74 | — |
| | 50 (° C.) | 7.31 | 6.41 | | | |
| EC 5.5% \| Oil 40% | 4 (° C.) | 7.60 | | 7.82 | 7.75 | 7.62 |
| | 25 (° C.) | 7.60 | | 6.97 | 5.78 | 5.51 |
| | 40 (° C.) | 7.60 | | 7.29 | 7.02 | — |
| | 50 (° C.) | 7.60 | 7.05 | | | |

TABLE 13
Accelerated pH Stability Results for Natural Oil Blend (avocado
oil (40% w/w), almond oil (30% w/w), and jojoba oil (30% w/w))

| Natural Blend | Temp (° C.) | Day 0 | Day 10 | Day 28 | Day 56 | Day 84 |
|---|---|---|---|---|---|---|
| EC 3.5% \| Oil 25% | 4 (° C.) | 8.12 | | 8.16 | 8.01 | 7.97 |
| | 25 (° C.) | 8.12 | | 6.93 | 5.96 | 5.99 |
| | 40 (° C.) | 8.12 | | 6.98 | 6.64 | 6.58 |
| | 50 (° C.) | 8.12 | 7.33 | | | |
| EC 3.5% \| Oil 25% | 4 (° C.) | 7.44 | | 7.45 | 7.44 | 6.99 |
| | 25 (° C.) | 7.44 | | 6.50 | 6.47 | 5.6 |
| | 40 (° C.) | 7.44 | | 6.47 | 6.79 | 6.66 |
| | 50 (° C.) | 7.44 | 6.28 | | | |
| EC 4% \| Oil 25% | 4 (° C.) | 4.06 | | — | 4.27 | — |
| | 25 (° C.) | 4.06 | | 4.17 | 4.35 | 4.29 |
| | 40 (° C.) | 4.06 | | 4.05 | 4.29 | 4.5 |
| | 50 (° C.) | 4.06 | 4.13 | | | |
| EC 4% \| Oil 25% | 4 (° C.) | 3.57 | | 3.84 | 3.78 | 3.99 |
| | 25 (° C.) | 3.57 | | 3.67 | 3.76 | 3.83 |
| | 40 (° C.) | 3.57 | | 3.68 | — | — |
| | 50 (° C.) | 3.57 | 3.59 | | | |
| SECB 4% \| Oil 10% | 4 (° C.) | 7.75 | | 7.82 | 7.71 | 7.5 |
| | 25 (° C.) | 7.75 | | 7.19 | 6.72 | 5.74 |
| | 40 (° C.) | 7.75 | | 6.75 | 7.03 | — |
| | 50 (° C.) | 7.75 | 6.76 | | | |
| EC 5% \| Oil 10% | 4 (° C.) | 7.67 | | 7.76 | 7.30 | 7.24 |
| | 25 (° C.) | 7.67 | | 6.75 | 6.53 | 5.86 |
| | 40 (° C.) | 7.67 | | 7.20 | 7.15 | 6.65 |
| | 50 (° C.) | 7.67 | 6.97 | | | |
| EC 5.5% \| Oil 40% | 4 (° C.) | 7.51 | | 7.2 | 7.81 | 6.53 |
| | 25 (° C.) | 7.51 | | 6.63 | 6.64 | — |
| | 40 (° C.) | 7.51 | | 6.7 | 5.87 | — |
| | 50 (° C.) | 7.51 | 6.98 | | | |

Viscosities were measured at 25° C. at 24 hours after making the emulsions using a rotational viscometer (Anton Paar, VISCOQC 100). The TRUEMODE program was used to run the measurements, which adjusts the speed so that a torque of approximately 80% is reached. The spindles used were RH-2 and RH3. Measurements were taken in triplicates and an average was reported. Tables 14-15 show the viscosity measurements at 24 hours for formulations made with the Example 5 emulsion composition and nonpolar emollients, squalane and mineral oil, respectively. The viscosity measurements for analogous formulation made with polar oils are presented in Tables 16-21 for the coco caprylate caprate, octyldodecanol, caprylic/capric triglyceride, sunflower oil, grapeseed oil, and natural oil blend emollients, respectively.

TABLE 14
Viscosity Results for Squalene at 25° C.

| | | Viscosity | Torque | Speed |
|---|---|---|---|---|
| Squalane | pH | (mPa · s) | (%) | (rpm) |
| EC 3.5% \| Oil 25% | 9.17 | 1102 | 76.9 | 44.7 |
| EC 3.5% \| Oil 25% | 5.22 | 2909 | 76.1 | 10.5 |
| EC 4% \| Oil 25% | 4.39 | 5794 | 75.7 | 5.3 |
| EC 4% \| Oil 25% | 3.58 | 9594 | 74.9 | 3.2 |
| EC 4% \| Oil 10% | 7.18 | 583 | 76.1 | 52.5 |
| EC 5% \| Oil 10% | 7.04 | 2337 | 76.5 | 13.1 |
| EC 5.5% \| Oil 40% | 7.56 | 24800 | 75.5 | 2.9 |

TABLE 15
Viscosity Results for Mineral Oil at 25° C.

| | | Viscosity | Torque | Speed |
|---|---|---|---|---|
| Mineral Oil | pH | (mPa · s) | (%) | (rpm) |
| EC 3.5% \| Oil 25% | 9.27 | 2419 | 76.6 | 13.9 |
| EC 3.5% \| Oil 25% | 7.64 | 5737 | 76.1 | 5.3 |
| EC 4% \| Oil 25% | 4.17 | 8696 | 74.5 | 3.7 |
| EC 4% \| Oil 25% | 3.81 | 6505 | 75.3 | 4.7 |
| EC 4% \| Oil 10% | 7.12 | 356 | 75 | 84.7 |
| EC 5% \| Oil 10% | 7.40 | 1799 | 76.4 | 17 |
| EC 5.5% \| Oil 40% | 7.84 | 4838 | 76.6 | 15.8 |

TABLE 16
Viscosity Results for Coco Caprylate Caprate at 25° C.

| Coco Caprylate | | Viscosity | Torque | Speed |
|---|---|---|---|---|
| Caprate | pH | (mPa · s) | (%) | (rpm) |
| EC 3.5% \| Oil 25% | 9.15 | 581 | 77.3 | 53.2 |
| EC 3.5% \| Oil 25% | 6.76 | 1049 | 76.8 | 29.3 |
| EC 4% \| Oil 25% | 3.94 | 2964 | 76.3 | 10.3 |
| EC 4% \| Oil 25% | 3.71 | 3729 | 76.4 | 8.2 |
| EC 4% \| Oil 10% | 7.18 | 311 | 76.1 | 98 |
| EC 5% \| Oil 10% | 7.04 | 1835 | 76.4 | 16.7 |
| EC 5.5% \| Oil 40% | 7.58 | 7554 | 76.3 | 10.1 |

TABLE 17
Viscosity Results for Octyldodecanol at 25° C.

| | | Viscosity | Torque | Speed |
|---|---|---|---|---|
| Octyldodecanol | pH | (mPa · s) | (%) | (rpm) |
| EC 3.5% \| Oil 25% | 9.23 | 974 | 76.6 | 31.5 |
| EC 3.5% \| Oil 25% | 7.14 | 2133 | 76.2 | 14.3 |
| EC 4% \| Oil 25% | 4.02 | 9113 | 74.6 | 3.5 |
| EC 4% \| Oil 25% | 3.76 | 9325 | 74.8 | 3.5 |
| EC 4% \| Oil 10% | 6.35 | 312 | 76.4 | 98 |
| EC 5% \| Oil 10% | 7.31 | 2165 | 76.3 | 14.1 |
| EC 5.5% \| Oil 40% | 7.60 | 9685 | 75.9 | 7.9 |

TABLE 18
Viscosity Results for Caprylic/Capric Triglyceride at 25° C.

| Caprylic/Capric | | Viscosity | Torque | Speed |
|---|---|---|---|---|
| Triglyceride | pH | (mPa · s) | (%) | (rpm) |
| EC 3.5% \| Oil 25% | 9.23 | 472 | 76.7 | 44.7 |
| EC 3.5% \| Oil 25% | 6.23 | 1273 | 76.2 | 24 |
| EC 4% \| Oil 25% | 4.41 | 12613 | 75.5 | 2.4 |
| EC 4% \| Oil 25% | 3.56 | 4573 | 76.2 | 6.7 |
| EC 3% \| Oil 10% | 7.12 | 87 | 70.9 | 66.4 |
| EC 5.5% \| Oil 40% | 7.54 | 9853 | 77.8 | 8 |

TABLE 19
Viscosity Results for Sunflower Oil at 25° C.

| | | Viscosity | Torque | Speed |
|---|---|---|---|---|
| Sunflower Oil | pH | (mPa · s) | (%) | (rpm) |
| EC 3.5% \| Oil 25% | 8.99 | 647 | 77.1 | 47.5 |
| EC 3.5% \| Oil 25% | 7.30 | 1464 | 76.5 | 20.9 |
| EC 4% \| Oil 25% | 4.14 | 5169 | 76.2 | 5.9 |
| EC 4% \| Oil 25% | 3.73 | 4996 | 75.8 | 6.5 |
| EC 3% \| Oil 10% | 6.43 | 81 | 72 | 73 |
| EC 5.5% \| Oil 40% | 6.42 | 16630 | 77.5 | 1.9 |

TABLE 20
Viscosity Results for Grapeseed Oil at 25° C.

| | | Viscosity | Torque | Speed |
|---|---|---|---|---|
| Grapeseed Oil | pH | (mPa · s) | (%) | (rpm) |
| EC 3.5% \| Oil 25% | 9.05 | 4382 | 75.8 | 7 |
| EC 3.5% \| Oil 25% | 7.26 | 2836 | 76.3 | 10.8 |
| EC 4% \| Oil 25% | 3.92 | 5565 | 76 | 5.5 |
| EC 4% \| Oil 25% | 3.71 | 8702 | 76.1 | 3.5 |
| EC 4% \| Oil 10% | 7.35 | 282 | 76.2 | 108 |
| EC 5% \| Oil 10% | 7.85 | 1482 | 76.3 | 20.9 |
| EC 5.5% \| Oil 40% | 7.34 | 7813 | 76.2 | 9.8 |

TABLE 21
Viscosity Results for Natural Oil Blend (avocado oil (40% w/w),
almond oil (30% w/w), and jojoba oil (30% w/w)) at 25° C.

| | | Viscosity | Torque | Speed |
|---|---|---|---|---|
| Natural Blend | pH | (mPa · s) | (%) | (rpm) |
| EC 3.5% \| Oil 25% | 8.12 | 601 | 77.0 | 51.9 |
| EC 3.5% \| Oil 25% | 7.44 | 767 | 76.7 | 40.3 |
| EC 4% \| Oil 25% | 4.06 | 2474 | 76.2 | 12.4 |
| EC 4% \| Oil 25% | 3.57 | 2930 | 76.0 | 10.3 |
| EC 4% \| Oil 10% | 7.75 | 244 | 76.3 | 125.0 |
| EC 5% \| Oil 10% | 7.67 | 1067 | 76.4 | 29.3 |
| EC 5.5% \| Oil 40% | 7.51 | 6250 | 76.4 | 12.2 |

In general, it was found that the Example 5 emulsifier composition does not affect the final viscosity of the emulsions significantly. This flexibility of viscosity allows the use of emulsifier compositions according to the disclosure for a wider range of applications. The viscosities of the emulsions were mainly dependent on the type of emollient and its concentration. The amount of emulsifier composition used also influenced the final viscosity of the emulsion but with a small impact. As in the case with the emollients, the higher the amounts of emulsifier used, the slightly higher the final viscosity. For some formulations, a higher viscosity was observed at lower pH values of about 3.5-4.0. Since the pKa of alginate is at 3.2, the alginates present in the sargassum extract could be changing into alginic acid and giving the emulsion the gel-like viscosity at pH levels close to the pKa value. The viscosity values in this example ranged as low as 77 mPa*s and as high as 24,800 mPa*s.

Emulsion Stability—Freeze-Thaw Cycling: The freeze-thaw cycling test determines the influence of different emollients, emollient concentrations, and emulsifier composition concentrations on the pH of emulsions over time. This stability test consists of subjecting emulsions to cycles of freezing and hot temperatures. The freeze-thaw cycle test is also considered an accelerated stability test for emulsions. For this test, emulsions are incubated at −20° C. for 24 hours; at t-24, emulsions are incubated at 40° C. for two hours. This freeze-thaw cycle is repeated up to three times and visual observations are recorded after each cycle. After the last thaw cycle, physical stability is determined by measuring the total height of the emulsion and the height of the upper phase separation. The following equation is used to determine emulsion's instability percentage: Emulsion instability (%)=(height of upper phase separation)/(total height of emulsion)×100%.

The emulsions with the highest aqueous phase content are more prone to destabilization and phase separation when subjected to repeated freeze-thaw cycles. This could be explained by the ice crystal formation in the aqueous phase leaving the oil phase (unfrozen phase) concentrated and pushing the oil droplets to coalesce. Also, it was observed that the Example 5 emulsifier concentrate is more effective in keeping the emulsions stable in neutral and alkaline pHs compared to acidic pHs. Table 22 shows the instability percentages for different emulsions.

TABLE 22
Emulsion Instability Percentage

| | Example 5 Emulsifier | | Emulsion |
|---|---|---|---|
| Oil Phase (wt. %) | Composition (wt. %) | pH Value | Instability (%) |
| Squalane 40% | 5.5% | 7 | 10.5 |
| Squalane 10% | 4% | 7 | 3.8 |
| Squalane 25% | 4% | 3.5 | 7.8 |
| Squalane 25% | 4% | 4 | 9 |
| Squalane 25% | 3.5% | 7 | 6.5 |
| Squalane 25% | 3.5% | 9 | 6.1 |
| Mineral Oil 40% | 5.5% | 7 | 12.2 |
| Mineral Oil 10% | 5% | 7 | 5.8 |
| Mineral Oil 10% | 4% | 7 | 3.2 |
| Mineral Oil 25% | 4% | 3.5 | 8.1 |
| Mineral Oil 25% | 4% | 4 | 10.5 |
| Mineral Oil 25% | 3.5% | 7 | 9 |
| Mineral Oil 25% | 3.5% | 9 | 8.2 |
| Caprylic/Capric Triglyceride 40% | 5.5% | 7 | 8.2 |
| Caprylic/Capric Triglyceride 10% | 3% | 7 | 2.4 |
| Caprylic/Capric Triglyceride 25% | 4% | 3.5 | 8 |
| Caprylic/Capric Triglyceride 25% | 4% | 4 | 7.2 |
| Caprylic/Capric Triglyceride 25% | 3.5% | 7 | 8.2 |
| Caprylic/Capric Triglyceride 25% | 3.5% | 9 | 7.3 |
| Coco Caprylate Caprate 40% | 5.5% | 7 | 12 |
| Coco Caprylate Caprate 10% | 4% | 7 | 4.2 |
| Coco Caprylate Caprate 25% | 4% | 3.5 | 7.8 |
| Coco Caprylate Caprate 25% | 4% | 4 | 8.6 |
| Coco Caprylate Caprate 25% | 3.5% | 7 | 6.6 |
| Coco Caprylate Caprate 25% | 3.5% | 9 | 4.6 |
| Sunflower Oil 40% | 5.5% | 7 | 11.9 |
| Sunflower Oil 10% | 3% | 7 | 3.8 |
| Sunflower Oil 25% | 4% | 3.5 | 7.2 |
| Sunflower Oil 25% | 4% | 4 | 8.1 |
| Sunflower Oil 25% | 3.5% | 7 | 6.6 |
| Sunflower Oil 25% | 3.5% | 9 | 6.7 |
| Grapeseed Oil 40% | 5.5% | 7 | 8 |
| Grapeseed Oil 10% | 5% | 7 | 2.8 |
| Grapeseed Oil 10% | 4% | 7 | 4 |
| Grapeseed Oil 25% | 4% | 3.5 | 9.2 |
| Grapeseed Oil 25% | 4% | 4 | 6.6 |
| Grapeseed Oil 25% | 3.5% | 7 | 6.8 |
| Grapeseed Oil 25% | 3.5% | 9 | 5.2 |

Emulsion Stability—Ionic Strength: The ionic strength test determines the influence of pH, ionic strength, and storage time on the stability of emulsions prepared with the Example 5 emulsion composition. The stability of emulsions made with OLIVEM 1000 (cetearyl olivate/sorbitan olivate), another natural emulsifier, was compared to the stability of the emulsions according to the disclosure. To determine the ionic strength while maintaining the stability of the emulsion, sodium chloride (NaCl) and calcium chloride (CaCl2) were added at different concentrations to formulations. The final formulations containing different concentrations of salts were 20% emollient; 2.8-3.2% emulsifier composition, 0.4% preservatives and water to complete 100%. The emulsions were incubated at room temperature and the time points studied were 1 day and 7 days. Final concentrations of NaCl in emulsions included: 0 mM, 50 mM, 100 mM, 250 mM, and 500 mM. Final concentrations of CaCl2 in emulsions included: 0 mM, 5 mM, 10 mM, 25 mM, and 50 mM. Emollients used for ionic strength test included: squalane, caprylic/capric triglyceride, coco caprylate caprate, sunflower oil, grapeseed oil, and mineral oil. Different pH values tested included: pH 9, 7, 4, and 3.5.

Ionic strength test results: None of the emulsions made with Example 5 emulsifier composition separated under the conditions tested. Squalane formulations (20% (w/w) squalane, 2.8-3.2% emulsifier, 0.4% pentylene glycol, and balance water) were incubated at room temperature in the presence of sodium chloride at different concentrations (0-500 mM). None of the emulsions showed signs of phase separation even after a 7-day incubation period. The emulsions remained stable even when exposed to environmental stress of high ionic strength (500 mM NaCl) and low pHs (3.5).

The same results were obtained with formulations exposed to different concentrations of calcium chloride, even at the highest concentration (50 mM CaCl2) tested. No phase separation was observed. The emulsions made at a pH of 3.5 showed a slight color change when exposed to the calcium salt. The emulsions at higher pHs and with or without salt exposure had a white color. However, emulsions at pH of 3.5 turned yellowish in color when they were exposed to calcium chloride. This color change was not observed in any of the emulsions exposed to sodium chloride.

In contrast, comparative emulsions formed using the commercial OLIVEM 1000 emulsifier resulted in phase separation when exposed to conditions representative of environmental stress such as pH and high sodium and calcium salt concentrations. The comparative formulations included 16% (w/w) squalane, 4% OLIVEM 1000, 0.4% pentylene glycol preservative, and balance water. Emulsions made with OLIVEM 1000 showed phase separation even after 1d of exposure to sodium and calcium salts.

Compatibility: The compatibility and performance of the Example 5 emulsifier composition with other ingredients commonly used in cosmetic formulations was also evaluated. The following components have tested with the Example 5 emulsifier composition and do not appear to adversely affect compatibility or performance of the emulsifier composition. Tested emollients include: caprylic/capric triglyceride, coco caprylate caprate, mineral oil, octyldodecanol, coconut oil, grapeseed oil, sunflower oil, squalane, silicones (e.g., dimethicone, silicone quarternium-3, trideceth-12, amodimethicone, c11-15 alketh-12, c11-15 alketh-7, cyclopentasiloxane), avocado oil, olive oil, jojoba oil, almond oil, sunflower wax, beeswax, shea butter, esters (e.g., cetyl esters, ethyl palmate, jojoba esters, octyldodecyl oleate, isopropyl palmitate, octyl palmitate), and limnanthes alba (meadowfoam) seed oil. Tested thickening agents include: xanthan gum, guar gum, konjac gum, sclerotium gum, cellulose and cellulose gum, carrageenan, alginates plus crosslinker, sargassum bioliquid extracts plus crosslinker, carbomers, cetearyl alcohol, glycerol stereate, glycerol stereate citrate, polyacrylates, and hydroxyethylcellulose. Tested preservatives include: pentylene glycol, phenoxyethanol, sodium levulinate, potassium sorbate, honeysuckle extract, caprylhydroxamic acid, sodium anisate, GERMALL PLUS (propylene glycol, diazolidinyl urea, iodopropynyl butylcarbamate), PLANTASERVE P (commercial preservative including phenoxyethanol), and ethanol. Tested chelating agents and anti-oxidants include: ethylenediaminetetraacetic acid (EDTA) and tocopherols. Tested active agents include: glycerin, isopropyl myristate, hyaluronic acid, glycol distearate, panthenol, niacinamide, ceramide, tetrahexyldecyl ascorbate, caprylhydroxamic acid, glyceryl caprylate, chamomile extract, and propanediol. Tested surfactants/coemulsifiers include: cocamidopropyl betaine, sodium myreth sulfate, decyl glucoside, hydroxysultaine, lauroyl sarcosinate, polyvinylpyrrolidone, sodium cocoyl isethionate, and PEG-100 stearate. Tested pigments, pearls, and colorants include: mineral, organic, pearls, titanium dioxide, and iron oxides. Tested UV filters include: titanium dioxide and zinc oxide. Tested pH modifiers or buffers include: lactic acid, citric acid, and sodium hydroxide.

Summary: The emulsifier compositions according to the disclosure, in particular the sargassum biosolid extracts containing a high-water content of up to 95%, have substantial emulsifying properties, being able to provide stable oil-in-water emulsions for a wide range of emollients, additional emulsion components, and emulsion properties (e.g., pH value, ionic concentration). Compatibility has been demonstrated for several thickening agents, including hydroxypropyltrimonium chloride, galactomannans, sclerotium gum, cellulose gum, alginates, carrageenans, sargassum bioliquid extracts, cetearyl alcohol, glycerol monostereate, glycerol stereate citrate, carbomer, and polyacrylates. The emulsifying efficacy of the biopolymeric complex present in the biosolids is directly proportional to the concentration of the bio complex. Based on these results, other sargassum extract-based emulsifier blends can be created, for example formulations containing up to 95% (w/w) sargassum extracts, thickening agents, and a preservative.

### Example 7—Formulation Examples

The following formulation examples illustrate different cosmetic compositions that can be formed with an emulsion composition according to the disclosure, for example the Example 5 emulsion composition above or otherwise.

Basic Lotion: A basic lotion can be formulated using the components listed in Table 23 using the following cold process procedure. Mix ingredients of Phase A in one vessel and homogenize until a consistent blend is obtained. Then add Phase B and continue homogenizing until it is completely dissolved. Add Phase C and homogenize until a uniform droplet size is created. Add Phase D to the emulsion and mix until it is homogeneous. The product is a white lotion with a viscosity of about 9500 cP (measured using an Anton Paar viscometer, RH4 Spindle, 5 rpm 5 mn). The viscosity of the basic lotion can be adjusted as desired with the addition of a suitable amount of a thickener (e.g., a selected thickener amount for a correspondence selected lotion viscosity). For example, thickened basic lotion formulations can be formed with the addition of cetearyl alcohol (11,000 cP final viscosity), glyceryl stereate (11,000 cP final viscosity), glyceryl stereate citrate (17,000 cP final viscosity), kappa and lambda carrageenan (25,650 cP final viscosity), a carbomer (32,600 cP final viscosity), guar gum (41,000 cP final viscosity), or konjac gum (63,730 cP final viscosity).

TABLE 23
Basic Lotion

| | Name | INCI Name | wt./wt. |
|---|---|---|---|
| A | Deionized Water | Water | q.s. 100% |
| | Citric Acid | Citric Acid | q.s. |
| | Example 5 Emulsifier | Sargassum Fluitans/Natans Extract, Xanthan | 4.0% |
| | Composition | Gum, Pentylene Glycol | |
| | Glycerin | Glycerin | 3.0% |
| B | Xanthan Gum | Xanthan Gum | 0.4% |
| C | Sunflower Oil | Helianthus Annuus (Sunflower) Seed Oil | 10.0% |
| D | Pentylene Glycol | Pentylene Glycol | 0.5% |

Sprayable Lotion: A sprayable lotion can be formulated using the components listed in Table 24 using the following cold process procedure. Mix ingredients of Phase A in one vessel and homogenize until a consistent blend is obtained. Then add Phase B and continue homogenizing until it is completely dissolved. Add Phase C and homogenize until a uniform droplet size is created. Add Phases D and E to the emulsion and mix until it is homogeneous.

TABLE 24
Sprayable Lotion

| | Trade Name | INCI Name | wt./wt. |
|---|---|---|---|
| A | Deionized Water | Water | q.s. 100% |
| | Citric Acid | Citric Acid | q.s. |
| | Example 5 Emulsifier | Sargassum Fluitans/Natans Extract, Xanthan | 2.0% |
| | Composition | Gum, Pentylene Glycol | |
| | Glycerin | Glycerin | 3.0% |
| B | CARBOPOL ULTREZ 10 | Carbomer | 0.15% |
| C | Sunflower Oil | Helianthus Annuus (Sunflower) Seed Oil | 15.0% |
| D | Pentylene Glycol | Pentylene Glycol | 0.5% |
| E | Sodium Hydroxide | Sodium Hydroxide | q.s. |

Body Milk: A body milk can be formulated using the components listed in Table 25 using the following cold process procedure. Mix ingredients of Phase A in one vessel and homogenize until a consistent blend is obtained. Then add Phase B and allow to hydrate, then continue homogenizing until it is completely dissolved. Add Phases C-E and homogenize until a uniform droplet size is created. Add Phase F to the emulsion and mix until it is homogeneous. The product is a white lotion with a viscosity of about 4400 cP (measured using an Anton Paar viscometer, RH4 Spindle, 5 rpm 5 mn).

TABLE 25
Body Milk

| | Trade Name | INCI Name | wt./wt. |
|---|---|---|---|
| A | Deionized Water | Water | q.s. 100% |
| | Citric Acid | Citric Acid | q.s. |
| | Example 5 Emulsifier | Sargassum Fluitans/Natans Extract, Xanthan | 2.0% |
| | Composition | Gum, Pentylene Glycol | |
| B | SEPIMAX ZEN | Polyacrylate Crosspolymer-6 | 0.60% |
| C | Golden Jojoba Oil | Simmondsia Chinensis (Jojoba) Seed Oil | 2.0% |
| D | LEXOL IPM | Isopropyl Myristate | 1.0% |
| E | BOTANISIL ME-14 PF | Silicone Quaternium-3, Trideceth-12 | 0.5% |
| F | EUXYL PE9010 | Phenoxyethanol (and) Ethylhexylglycerin | 1.0% |

Hydrating Serum: A hydrating serum can be formulated using the components listed in Table 26 using the following cold process procedure. Mix ingredients of Phase A in one vessel and homogenize until a consistent blend is obtained. Then add Phases B and C and homogenize until a uniform droplet size is created. Add Phases D-E to the emulsion and mix until it is homogeneous.

TABLE 26
Hydrating Serum

| | Trade Name | INCI Name | wt./wt. |
|---|---|---|---|
| A | Deionized Water | Water | q.s. 100% |
| | Citric Acid | Citric Acid | q.s. |
| | Example 5 Emulsifier | Sargassum Fluitans/Natans Extract, Xanthan | 2.0% |
| | Composition | Gum, Pentylene Glycol | |
| | Glycerin | Glycerin | 3.0% |
| B | Squalane | Squalane | 3.0% |
| C | Golden Jojoba Oil | Simmondsia Chinensis (Jojoba) Seed Oil | 1.0% |
| D | EUXYL PE9010 | Phenoxyethanol (and) Ethylhexylglycerin | 1.0% |
| E | Hyaluronic Acid 1% | Water and Sodium Hyaluronate | 2.0% |

Gentle Creamy Cleanser: A gentle creamy cleanser can be formulated using the components listed in Table 27 using the following hot process procedure. Mix ingredients of Phase A in one vessel and homogenize until a consistent blend is obtained. Then add Phase B and continue homogenizing until it is completely dissolved. Begin heating to 80° C. Premix ingredients of Phase C in a side vessel and heat to 80° C. When both mixtures reach temperature, pour the side vessel into the main vessel, and homogenize until a uniform droplet size is created. Begin cooling down and add Phase 2 and continue homogenization until fully blended. Add Phase E to the emulsion and mix until it is homogeneous. The product is a white lotion with a viscosity of about 3600 cP (measured using a Brookfield RV DV-L+P viscometer, Sp 4.10 rpm 30 sec).

TABLE 27
Gentle Creamy Cleanser

| | Trade Name | INCI Name | wt./wt. |
|---|---|---|---|
| A | Deionized Water | Water | to 100% |
| | Citric Acid | Citric Acid | q.s. |
| | Example 5 Emulsifier | Sargassum Fluitans/Natans Extract, | 1.0% |
| | Composition | Xanthan Gum, Pentylene Glycol | |
| | Glycerin | Glycerin | 2% |
| B | KELTROL T | Xanthan Gum | 0.40% |
| C | Golden Jojoba Oil | Simmondsia Chinensis (Jojoba) Seed Oil | 4.0% |
| | LANETTE O NA MB | Cetearyl Alcohol | 4.0% |
| D | CHEMBETAINE C | Water, Cocamidopropyl Betaine | 15% |
| E | EUXYL PE9010 | Phenoxyethanol, Ethylhexylglycerin | 1.0% |

Sunscreen Emulsion: A sunscreen emulsion with mineral filters (SPF30) can be formulated using the components listed in Table 28 using the following hot process procedure. Mix ingredients of Phase A in one vessel and homogenize until a consistent blend is obtained. Mix ingredients of Phase B in a side vessel and homogenize. Begin heating Phase A and Phase B to 75° C. When both mixtures reach temperature, pour the side vessel into the main vessel, and homogenize until a uniform droplet size is created. Begin cooling down to 45° C. and add Phase C and add Phase C until it is homogeneous. The product is a white cream with a pH in a range of about 7.5-8.0, an M3 stability rating, and a viscosity of about 55000-65000 cP, (measured using a Brookfield RV viscometer, 5 rpm 1 mn).

TABLE 28
Sunscreen Emulsion

| | Trade Name | INCI Name | wt./wt. |
|---|---|---|---|
| A | Deionized Water | Water | to 100% |
| | Example 5 Emulsifier | Sargassum Fluitans/Natans Extract, | 1.5% |
| | Composition | Xanthan Gum, Pentylene Glycol | |
| | BETAFIB ETD | Cellulose (and) Cellulose Gum | 0.9% |
| | Glycerin | Glycerin | 5% |
| | EDTA | Disodium EDTA | 0.2% |
| B | ZETEMULS SMS | Sorbitan Stearate | 3% |
| | Dicaprylyl Ether | Dicaprylyl Ether | 5% |
| | HELIOPRO ZN TG 60 | Caprylic Capric Triglyceride, Zinc Oxide | 36% |
| | | (nano), Polyglyceryl-3 Polyricinoleate | |
| | HELIOPRO TG 50 H | Caprylic/Capric Triglyceride, Titanium | 6% |
| | | Dioxide, Polyhydroxystearic Acid, | |
| | | Aluminum Stearate, Alumina | |
| | SORBOSIL TC 15 | Hydrated Silica | 2% |
| C | Preservative | — | 1% |
| | Vitamin E Acetate | Tocopheryl Acetate | 1% |
| | Fragrance | — | 0.3% |

Moisturizing Cream: Moisturizing creams can be formulated using the components listed in Table 29 for each of Cream 1, Cream 2, and Cream 3 using the following hot process procedure. Mix ingredients of Phase A in one vessel and homogenize until a consistent blend is obtained. Mix ingredients of Phase B in a side vessel and homogenize. Begin heating Phase A and Phase B to 75° C. When both mixtures reach temperature, pour the side vessel into the main vessel, and homogenize until a uniform droplet size is created. Begin cooling down to 45° C. and add Phase C under moderate stirring. The products are white creams with a pH in a range of about 5.0-5.5. Cream 1 has a viscosity of about 3500-4000 cP,(1 week storage), 3000-4000 cP,(1 month storage), and 3500-4000 cP,(3 months storage). Cream 2 has a viscosity of about 6500-7000 cP,(1 week storage), 6000-6500 cP,(1 month storage), and 6000-6500 cP,(3 months storage). Cream 3 has a viscosity of about 10000-12000 cP,(1 week storage), 10000-12000 cP,(1 month storage), and 12000-12500 cP,(3 months storage). Each viscosity measurement is made using a Brookfield RV viscometer, 5 rpm 1 mn.

TABLE 29
Moisturizing Cream

| | | | Cream 1 | Cream 2 | Cream 3 |
|---|---|---|---|---|---|
| | Trade Name | INCI Name | wt./wt. | wt./wt. | wt./wt. |
| A | Deionized Water | Water | to 100% | to 100% | to 100% |
| | Glycerin | Glycerin | 3% | 3% | 3% |
| | Example 5 Emulsifier | Sargassum Fluitans/Natans | 1.5% | 3% | 5% |
| | Composition | Extract, Xanthan Gum, | | | |
| | | Pentylene Glycol | | | |
| | Xanthan Gum | Xanthan Gum | 0.3% | 0.3% | 0.3% |
| B | Cetearyl Alcohol | Cetearyl Alcohol | 1.5% | 1.5% | 1.5% |
| | DERMOL HDE | Isohexadecane, PPG-15 | 5% | 5% | 5% |
| | | Stearyl Ether | | | |
| | SYMMOLLIENT PDCC | Propanediol | 6% | 6% | 6% |
| | | Dicaprylate/Caprate | | | |
| | SYMMOLLIENT S | Cetearyl Nonanoate | 2% | 2% | 2% |
| | GREEN | | | | |
| | Prickly Pear Seed Oil | Opuntia Ficus-indica Seed Oil | 1.5% | 1.5% | 1.5% |
| C | Preservative & | — | qs | qs | qs |
| | Fragrance | | | | |

Lightweight Cream: lightweight creams can be formulated using the components listed in Table 30 using the following cold process procedure. Mix ingredients of Phase A in one vessel and homogenize until a consistent blend is obtained. Phase B is then added to Phase A and homogenized. Phase C is added and homogenized. The products are white creams with a pH in a range of about 4.0-4.5. Cream has a viscosity of about 6000-7000 cP. Each viscosity measurement is made using a Brookfield RV viscometer, 5 rpm 1 mm

TABLE 30
Lightweight Cream

| PHASE | SEQ | TRADE NAME | INCI | % |
|---|---|---|---|---|
| A | 1 | Deionized Water | Water | 91.85% |
| A | 2 | Citric Acid USP | Citric Acid | 0.05% |
| A | 3 | SEPIMAX ZEN | Polyacrylate Crosspolymer-6 | 0.60% |
| A | 4 | EUXYL PE 9010 | Phenoxyethanol, Ethylhexylglycerin | 1.00% |
| A | 5 | Example 5 | Pentylene Glycol, Sargassum Fluitans/Natans | 3.00% |
| | | Emulsifier | Extract, Xanthan Gum | |
| | | Composition | | |
| B | 6 | Jojoba Seed Oil | Simmondsia Chinensis (Jojoba) Seed Oil | 2.00% |
| B | 7 | LEXOL IPM | Isopropyl Myristate | 1.00% |
| B | 8 | XIAMETER PMX- | Dimethicone | 0.50% |
| | | 200 100 cst | | |

Hair Styling Lotion: hair styling lotions can be formulated using the components listed in Table 31 using the following cold process procedure. Mix ingredients of Phase A one-by-one in one vessel and homogenize until a consistent blend is obtained. Phase B is then added to Phase A and homogenized. Phase C is added under moderate mixing. The products have a pH in a range of about 5.0-5.5. Lotion has a viscosity of about 2500-3500 cP. Each viscosity measurement is made using a Brookfield RV viscometer, 5 rpm 1mn.

TABLE 31
Hair Styling Lotion

| PHASE | SEQ | TRADE NAME | INCI | % |
|---|---|---|---|---|
| A | 1 | Deionized Water | Water | 92.85% |
| A | 2 | Citric Acid USP | Citric Acid | 0.05% |
| A | 3 | DL Panthenol | Panthenol | 0.50% |
| A | 4 | NATROSOL 250HHR | Hydroxyethylcellulose | 0.10% |
| A | 5 | EUXYL PE 9010 | Phenoxyethanol, Ethylhexylglycerin | 1.00% |
| A | 6 | Example 5 | Pentylene Glycol, Sargassum Fluitans/Natans | 3.00% |
| | | Emulsifier | Extract, Xanthan Gum | |
| | | Composition | | |
| A | 7 | DC 979 Emulsion | Amodimethicone, C11-15 Alketh-12, C11-15 | 2.00% |
| | | | Alketh-7 | |
| A | 8 | Meadowfoam Seed | Limnanthes Alba (Meadowfoam) Seed Oil | 1.00% |
| | | Oil | | |
| A | 9 | PVP K-90 20% | Water, PVP | 0.50% |
| | | Soln | | |

BB Cream: Beautifying balm (BB) creams can be formulated using the components listed in Table 32 using the following hot process procedure. Charge vessel with #1-3 and begin moderate mixing. Add #5 and homogenize until smooth. Begin heating to 75-80° C.

Premix #6-8 and heat to 75-80° Add to main vessel once melted and homogenize until a smooth, milky liquid is obtained. Begin cooling under propeller mixing to 40° C. At 40° C., add #9-13 and homogenize until all pigments are dispersed. Pigments may need to be adjusted depending on desired tonal shade. This shade is a deep warm, golden shade with red undertones. The products have a pH in a range of about 4.5-5.5. BB cream has an initial viscosity of about 2500-3500 cP (time=0) and a final viscosity in the 18000-22000 cP. Each viscosity measurement is made using a Brookfield RV viscometer, 5 rpm 1 mn.

TABLE 32
BB Cream

| PHASE | SEQ | TRADE NAME | INCI | % |
|---|---|---|---|---|
| A | 1 | Deionized Water | Water | 63.95% |
| A | 2 | Citric Acid USP | Citric Acid | 0.05% |
| A | 3 | Zemea Propanediol | Propanediol | 5.00% |
| B | 4 | Example 5 | Pentylene Glycol, Sargassum Fluitans/Natans | 3.50% |
| | | Emulsifier | Extract, Xanthan Gum | |
| | | Composition | | |
| B | 5 | EUXYL PE 9010 | Phenoxyethanol, Ethylhexylglycerin | 1.00% |
| C | 6 | ARLACEL 165 | Glyceryl Stearate (and) PEG-100 Stearate | 2.00% |
| C | 7 | LANETTE O | Cetearyl Alcohol | 2.00% |
| C | 8 | LIPEX SHEA | Butyrospermum Parkii (Shea) Butter | 10.00% |
| D | 9 | XIAMETER PMX- | Cyclopentasiloxane | 10.00% |
| | | 0245 | | |
| D | 10 | OLIVSPERSE | Titanium Dioxide, Octyldodecyl Oleate | 6.00% |
| | | WHITE | | |
| D | 11 | OLIVSPERSE | Iron Oxides, Octyldodecyl Oleate | 3.00% |
| | | YELLOW IO | | |
| D | 12 | OLIVSPERSE RED | Iron Oxides, Octyldodecyl Oleate | 0.80% |
| | | IO | | |
| D | 13 | OLIVSPERSE | Iron Oxides, Octyldodecyl Oleate | 0.26% |
| | | BLACK IO | | |

Anti-Aging Cream: anti-aging creams can be formulated using the components listed in Table 33 using the following hot process procedure. Charge vessel with #1 and begin moderate mixing. Add #2. When dissolved, increase mixing speed to generate a strong vortex and add #4 slowly by sprinkling it into the vortex. Mix until completely dispersed. Add #4-5. Begin heating to 75-80° C. In a side vessel, premix Phase C and begin heating to 75-80° C. When both are at temperature, pour Phase C into Phase A/B and homogenize until completely dispersed. Begin cooling to 40° C. under moderate propeller mixing. Add #13. Measure pH and adjust pH to 5.50-6.00 using an 18% sodium hydroxide solution, if necessary. The products have a pH in a range of about 5.5-6.0. Anti-aging cream has an initial viscosity of about 6500-7500 cP (time=0), a viscosity in the 14000 cP (time=24 hour), and a viscosity in the 115000 cP (time=2 weeks). Each viscosity measurement is made using a Brookfield RV viscometer, 5 rpm 1 mn, except for the high final viscosity where Sp. TE, and 30 second with Helipath down was used.

TABLE 33
Anti-Aging Cream

| PHASE | SEQ | TRADE NAME | INCI | % |
|---|---|---|---|---|
| A | 1 | Deionized Water | Water | 66.40% |
| A | 2 | Citric Acid USP | Citric Acid | 0.05% |
| A | 3 | CARBOPOL ULTREZ 20 | Acrylates/C10-30 Alkyl Acrylate | 0.15% |
| | | | Crosspolymer | |
| A | 4 | Glycerin 99.7% USP | Glycerin | 3.00% |
| A | 5 | Niacinamide USP | Niacinamide | 2.00% |
| B | 6 | Example 5 Emulsifier | Pentylene Glycol, Sargassum Fluitans/Natans | 4.00% |
| | | Composition | Extract, Xanthan Gum | |
| C | 7 | LANETTE O | Cetearyl Alcohol | 2.00% |
| C | 8 | MIGLYOL 812 | Caprylic/Capric Triglyceride | 10.00% |
| C | 9 | LIPEX SHEA | Butyrospermum Parkii (Shea) Butter | 10.00% |
| C | 10 | DS-HYDROCERAMIDE | Ceramide NP | 0.20% |
| | | 50 | | |
| C | 11 | BV-OSC | Tetrahexyldecyl Ascorbate | 0.50% |
| C | 12 | XIAMETER PMX-200 | Dimethicone | 0.20% |
| | | 100 cst | | |
| D | 13 | SPECTRASTAT G2 N | Caprylhydroxamic Acid, Glyceryl Caprylate, | 1.50% |
| | | MB | Glycerin | |
| D | 14 | NaOH 18% Solution | Sodium Hydroxide | Q.S. |

Oil-Free Cream: oil-free creams can be formulated using the components listed in Table 34 using the following hot process procedure. Charge vessel with #1 and begin moderate mixing. Add #2-4. When dissolved, add #5 and homogenize. Begin heating to 75-80° C. under gentle mixing. In a side vessel, premix Phase C and begin heating to 75-80° C. When both are at temperature, pour Phase C into Phase A/B and homogenize until completely dispersed. Begin cooling to 40° C. under moderate propeller mixing. Add #10. The products have a pH in a range of about 4.5-5.0. Oil-free cream has an initial viscosity of about 1500-2500 cP (time=0) and a final viscosity of 5000-6000 cP (time=2 weeks). Each viscosity measurement is made using a Brookfield RV viscometer, 5 rpm 1mn.

TABLE 34
Oil-Free Cream

| PHASE | SEQ | TRADE NAME | INCI | % |
|---|---|---|---|---|
| A | 1 | Deionized Water | Water | 74.45% |
| A | 2 | Citric Acid USP | Citric Acid | 0.05% |
| A | 3 | DERMATEIN HYA 1% | Water, Hyaluronic Acid | 3.00% |
| A | 4 | EUXYL PE 9010 | Phenoxyethanol, Ethylhexyl Glycerin | 1.00% |
| B | 5 | Example 5 Emulsifier | Pentylene Glycol, Sargassum Fluitans/Natans | 5.00% |
| | | Composition | Extract, Xanthan Gum | |
| C | 6 | LANETTE 18 | Stearyl Alcohol | 2.00% |
| C | 7 | Cetyl Esters | Cetyl Esters | 2.00% |
| C | 8 | ALLYESTER | Ethyl Palmate | 7.00% |
| C | 9 | FLORAESTERS 15 | Jojoba Esters | 5.00% |
| D | 10 | Chamomile Extract | Anthemis Nobilis Flower Extract | 0.50% |

Natural Lotion: natural lotions can be formulated using the components listed in Table 35 using the following hot process procedure. Charge vessel with #1 and begin moderate mixing. Add #2. Premix #3-4 and add slurry to main vessel, When dispersed appropriately, batch will become clear. Add #5-6 and homogenize until smooth. Begin heating to 75-80° C. under gentle mixing. In a side vessel, premix Phase C and begin heating to 75-80° C. When both are at temperature, pour Phase C into Phase A/B and homogenize until completely dispersed. Begin cooling to 40° C. under moderate propeller mixing. The products have a pH in a range of about 4.5-5.0. Natural lotion has a final viscosity of 5000-6000 cP (time=2 weeks). Each viscosity measurement is made using a Brookfield RV viscometer, 5 rpm 1 mn.

TABLE 35
Natural Lotion

| PHASE | SEQ | TRADE NAME | INCI | % |
|---|---|---|---|---|
| A | 1 | Deionized Water | Water | 74.45% |
| A | 2 | Citric Acid USP | Citric Acid | 0.05% |
| A | 3 | COSPHADERM X34 | Xanthan Gum | 0.40% |
| A | 4 | Glycerin 99.7% USP | Glycerin | 3.00% |
| B | 5 | Example 5 Emulsifier | Pentylene Glycol, Sargassum Fluitans/Natans | 4.00% |
| | | Composition | Extract, Xanthan Gum | |
| B | 6 | EUXYL PE 9010 | Phenoxyethanol, Ethylhexylglycerin | 1.00% |
| C | 7 | LANETTE O | Cetearyl Alcohol | 7.20% |
| C | 8 | Sunflower Seed Oil | Helianthus Annuus (Sunflower) Seed Oil | 10.00% |

Because other modifications and changes varied to fit particular operating requirements and environments will be apparent to those skilled in the art, the disclosure is not considered limited to the example(s) chosen for purposes of illustration, and covers all changes and modifications which do not constitute departures from the true spirit and scope of this disclosure.

Accordingly, the foregoing description is given for clearness of understanding only, and no unnecessary limitations should be understood therefrom, as modifications within the scope of the disclosure may be apparent to those having ordinary skill in the art.

All patents, patent applications, government publications, government regulations, and literature references cited in this specification are hereby incorporated herein by reference in their entirety. In the case of conflict, the present description, including definitions, will control.

Throughout the specification, where the compounds, compositions, methods, and/or processes are described as including components, steps, or materials, it is contemplated that the compounds, compositions, methods, and/or processes can also comprise, consist essentially of, or consist of any combination of the recited components or materials, unless described otherwise. Component concentrations can be expressed in terms of weight concentrations, unless specifically indicated otherwise. Combinations of components are contemplated to include homogeneous and/or heterogeneous mixtures, as would be understood by a person of ordinary skill in the art in view of the foregoing disclosure.

1. A method for extracting an alginate biomass, the method comprising:
providing an alginate-containing biomass pulp;
performing a first extraction process comprising:
adding a base selected from the group consisting of inorganic acid salts and hydroxides, and optionally water in amounts sufficient to the biomass pulp to provide a biomass dough having (i) a water content in a range of 40-80 wt. %, (ii) a base content in a range of 5-20 wt. %, and (iii) a solids content in a range of 15-55 wt. % based on the biomass dough; and
maintaining the biomass dough at a temperature up to 35° C. for a time sufficient to convert alginate originally present in the biomass pulp into a water-soluble alginate salt; and
performing a second extraction process comprising:
adding water in an amount sufficient to the biomass dough containing the water-soluble alginate salt to provide a biomass suspension having (i) a water content in a range of 80-98 wt. %, and (ii) a solids content in a range of 2-20 wt. % based on the biomass suspension; and
partitioning the biomass suspension at a temperature up to 35° C. for a time sufficient to form a bioliquids phase and a separate biosolids phase therein, wherein (i) the bioliquids phase comprises water and at least a portion of the water-soluble alginate salt dissolved therein, and (ii) the biosolids phase comprises proteins, optionally amino acids, cellulose, and at least a portion of the water-soluble alginate salt extracted from the biomass pulp.

2-30. (canceled)

31. A biosolids material formed according to claim 1.

32. A bioliquids material formed according to claim 1.

33. An emulsifier composition comprising:
at least one of a biosolids and a bioliquids alginate biomass extract;
a thickening agent; and
optionally a preservative.

34. The emulsifier composition of claim 33, wherein:
the emulsifier composition comprises the biosolids alginate biomass extract; and
the biosolids alginate biomass extract is present in an amount in a range of 50 to 98 wt. % relative to the emulsifier composition;
the thickening agent is present in an amount in a range of 2 to 40 wt. % relative to the emulsifier composition;
the thickening agent is selected from the group consisting of xanthan gum, guar gum, koniac gum, sclerotium gum, cellulose and cellulose gum, carraqeenan, alginates plus crosslinker, crosslinked sarqassum bioliquid extracts, carbomers, cetearyl alcohol, glycerol stereate, glycerol stereate citrate, polyacrylates, hydroxyethylcellulose, and combinations thereof;
the preservative is present in an amount in a range of 1 to 20 wt. % relative to the emulsifier composition; and
the preservative is selected from the group consisting of pentylene glycol, phenoxyethanol, sodium levulinate, potassium sorbate, honeysuckle extract, caprylhydroxamic acid, sodium anisate, propylene glycol, diazolidinyl urea, iodopropynyl butylcarbamate, ethanol, and combinations thereof.

35-36. (canceled)

37. The emulsifier composition of claim 33, wherein:
the emulsifier composition comprises the biosolids alginate biomass extract, which is a sargassum extract and is present in an amount in a range of 80 to 95 wt. % relative to the emulsifier composition;
the thickening agent comprises xanthan gum, which is present in an amount in a range of 3 to 15 wt. % relative to the emulsifier composition; and
the preservative is present and comprises pentylene glycol, which is present in an amount in a range of 1 to 8 wt. % relative to the emulsifier composition.

38. The emulsifier composition of claim 33, wherein:
the alginate biomass extract comprises a sargassum biomass extract; and
the alginate biomass extract comprises a polymer complex comprising alginate, protein, and cellulose bound together.

39. The emulsifier composition of claim 33, wherein the alginate biomass extract is a biosolids alginate biomass extract comprising;
alginate;
at least 40 wt. % cellulose relative to total organic content of the biosolids alginate biomass extract;
5 wt. % to 40 wt. % protein relative to total organic content of the biosolids alginate biomass extract;
a polymer complex comprising at least a portion of the alginate, the protein, and the cellulose bound together; and
optionally, one or more of non-cellulose fibers, fatty acids, and free amino acids.

40. The emulsifier composition of claim 33, wherein the alginate biomass extract is a bioliquids alginate biomass extract.

41. The emulsifier composition of claim 33, wherein:
the emulsifier composition has a pH value in a range of 7 to 11; and
the emulsifier composition has a viscosity value in a range of 200 to 2000 cP, as measured in a 10 wt. % aqueous solution of the emulsifier composition at 25° C.

42. (canceled)

43. An emulsion comprising:
water;
oil; and
the emulsifier composition of claim 33.

44. The emulsion of claim 43, wherein:
the emulsion is in the form of an oil-in-water emulsion;
the water is present in an amount in a range of 50 to 98 wt. % relative to the emulsion;
the oil is present in an amount in a range of 2 to 50 wt. % relative to the emulsion;
the emulsifier composition is present in an amount of 0.2 to 20 wt. % relative to the emulsion; and
a ratio of emulsifier composition to oil present in the emulsion is in a range of 1:1.5 to 1:10 (w/w).

45. (canceled)

46. The emulsion of claim 43, wherein the oil comprises an emollient selected from the group consisting of caprylic/capric triglyceride, coco caprylate caprate, mineral oil, octyldodecanol, grapeseed oil, sunflower oil, squalane, avocado oil, jojoba oil, almond oil, and combinations thereof.

47. The emulsion of claim 43, wherein:
the oil has a polarity index in a range of 15 mN/m to 50 mN/mi
the emulsion comprises one or more further components selected from the group consisting of preservatives, thickening agents, chelating agents, anti-oxidants, active agents, surfactants, pigments, colorants, UV filters, pH-adjusting agents, and combinations thereof;
the emulsion has a pH value in a range of 3 to 11; and
the emulsion has viscosity value in a range of 50 to 100000 cP, as measured at 25° C.

48-50. (canceled)

51. The emulsion of claim 43, wherein:
the emulsion has a pH value that remains within 1.5 pH units of its original pH value upon formation for a period of 10 to 84 days at a storage temperature of 4° C. to 40° C.;
the emulsion has an emulsion instability of 15% or less when subjected to a freeze-thaw cycle test;
the emulsion remains stable after formation for a period of 7 days when combined with NaCl at a concentration of 500 mM in the emulsion; and
the emulsion remains stable after formation for a period of 7 days when combined with CaCl₂ at a concentration of 50 mM in the emulsion.

52-54. (canceled)

55. The emulsion of claim 43, wherein the emulsion is in the form of a cosmetic composition selected from the group consisting of a lotion, a cream, a body milk, and a hydrating serum.

56-68. (canceled)

69. A method for extracting an alginate biomass, the method comprising:
providing an alginate-containing biomass pulp;
performing a first extraction process comprising:
adding a base selected from the group consisting of inorganic acid salts and hydroxides, and optionally water in amounts sufficient to the biomass pulp to provide a biomass dough; and
maintaining the biomass dough at a first temperature for a time sufficient to convert alginate originally present in the biomass pulp into a water-soluble alginate salt; and
performing a second extraction process comprising:
adding water in an amount sufficient to the biomass dough containing the water-soluble alginate salt to provide a biomass suspension; and
partitioning the biomass suspension at a second temperature for a time sufficient to form a bioliquids phase and a separate biosolids phase therein, wherein (i) the bioliquids phase comprises water and at least a portion of the water-soluble alginate salt dissolved therein, and (ii) the biosolids phase comprises proteins, optionally amino acids, cellulose, and at least a portion of the water-soluble alginate salt extracted from the biomass pulp.

70-72. (canceled)

73. The emulsion of claim 43, further comprising:
a surfactant present in an amount of 0.1 to 7 wt. % relative to the emulsion

74. The emulsion of claim 43, wherein the emulsifier composition comprises a biosolids alginate biomass extract as the alginate biomass extract, the biosolids alginate biomass extract comprising:
alginate;
at least 40 wt. % cellulose relative to total organic content of the biosolids alginate biomass extract;
5 wt. % to 40 wt. % protein relative to total organic content of the biosolids alginate biomass extract;
optionally, one or more of non-cellulose fibers, fatty acids, and free amino acids.

75. The emulsion of claim 74, wherein:
the emulsion is in the form of an oil-in-water emulsion; and
at least a portion of the cellulose is adsorbed at an oil-water interface in the emulsion to form a coating around droplets of the oil, thereby (i) providing a mechanical barrier to coalescence of the droplets of the oil, and (ii) stabilizing the emulsion.
